SEQUENCE LISTING (28 sequences)

SEQ ID NO: 1 - protein, 5 aa, synthetic construct
SYDMS

SEQ ID NO: 2 - protein, 17 aa, synthetic construct
GIYSGDGSIYYADSVKG

SEQ ID NO: 3 - protein, 12 aa, synthetic construct
DGPMNEESRFDY

SEQ ID NO: 4 - protein, 13 aa, synthetic construct
SGSSSNIGNNYVS

SEQ ID NO: 5 - protein, 7 aa, synthetic construct
ADSKRPS

SEQ ID NO: 6 - protein, 11 aa, synthetic construct
ASWDYSLSGYV

SEQ ID NO: 7 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPMNEESRFDYWGQGTLVTVSS

SEQ ID NO: 8 - protein, 111 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLSGYVFGGGTKLTVLG

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 11 - protein, 104 aa, synthetic construct
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 12 - protein, 451 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPMNEESRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 449 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPMNEESRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 14 - protein, 215 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 15 - protein, 457 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPMNEESRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGALPETGG

SEQ ID NO: 16 - protein, 223 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGALPETGG

SEQ ID NO: 17 - protein, 5 aa, synthetic construct
GGGGS

SEQ ID NO: 18 - protein, 10 aa, synthetic construct
GGGGSGGGGS

SEQ ID NO: 19 - protein, 15 aa, synthetic construct
GGGGSGGGGSGGGGS

SEQ ID NO: 20 - protein, 6 aa, synthetic construct
  VARIANT 3: note = X at position 3 is any single amino acid that is natural or unnatural
  VARIANT 6: note = X at position 6 is absent, or is an amino acid fragment comprising 1-10 amino acids
LPXTGX

SEQ ID NO: 21 - protein, 5 aa, synthetic construct
  VARIANT 3: note = X at position 3 is any single amino acid that is natural or unnatural
LPXTG

SEQ ID NO: 22 - protein, 6 aa, synthetic construct
LPETGG

SEQ ID NO: 23 - protein, 5 aa, synthetic construct
NPQTN

SEQ ID NO: 24 - protein, 5 aa, synthetic construct
NPKTG

SEQ ID NO: 25 - protein, 5 aa, synthetic construct
  VARIANT 3: note = X at position 3 is any single amino acid that is natural or unnatural
LAXTG

SEQ ID NO: 26 - protein, 7 aa, synthetic construct
LPQTSEQ

SEQ ID NO: 27 - protein, 4 aa, synthetic construct
GGFG

SEQ ID NO: 28 - protein, 5 aa, synthetic construct
LPETG

(19) United States
(12) Patent Application Publication
(10) Pub. No.: US 2025/0387505 A1
(43) Pub. Date: Dec. 25, 2025

(54) ANTI-FGFR3 ANTIBODY CONJUGATE AND MEDICAL USE THEREOF

(71) Applicants: GENEQUANTUM HEALTHCARE (SUZHOU) CO., LTD., Suzhou (CN); AIMED BIO INC., Seoul (KR)

(72) Inventors: Gang QIN, Suzhou (CN); Paul H. SONG, Suzhou (CN); Byeongkwi MIN, Seoul (KR); Mingyu HU, Suzhou (CN)

(21) Appl. No.: 18/879,386

(22) PCT Filed: Jun. 28, 2023
(86) PCT No.: PCT/CN2023/103152

§ 371 Date: Dec. 27, 2024

(30) Foreign Application Priority Data
Jun. 28, 2022 (WO) PCT/CN2022/101787
Mar. 13, 2023 (WO) PCT/CN2023/081140
Apr. 13, 2023 (WO) PCT/CN2023/088092

(51) Int. Cl. A61K 47/68; A61P 35/00; C07K 16/28

(52) U.S. Cl. CPC A61K 47/6849; A61K 47/68037; A61P 35/00; C07K 16/2863; C07K 2317/73; C07K 2317/77; C07K 2317/92

(57) ABSTRACT

Provided is related to the biopharmaceutical field, in particular, conjugates containing anti-FGFR3 antibodies and linker-payloads, and the corresponding pharmaceutical composition, preparing process and use thereof.

### CROSS REFERENCE TO RELATED APPLICATIONS

This application is a National Stage Entry under 35 U.S.C. § 371 of International Application No. PCT/CN2023/103152 filed Jun. 28, 2023, which claims the benefit of International Application Nos. PCT/CN2023/088092 filed Apr. 13, 2023, PCT/CN2023/081140 filed Mar. 13, 2023, and PCT/CN2022/101787 filed Jun. 28, 2022, the contents of each of which are incorporated herein by reference.

### REFERENCE TO A SEQUENCE LISTING

This application contains a Sequence Listing as a separate part of the disclosure. The contents of the Sequence Listing (2024-12-22-GQH-AIM-21-US-Sequence Listing.xml; Size: 29,696 bytes; and Date of Creation: Dec. 12, 2024) are incorporated herein by reference.

### TECHNICAL FIELD

The present disclosure relates to the biopharmaceutical field, in particular, conjugates containing anti-FGFR3 antibodies and linker-payloads, and the corresponding pharmaceutical composition, preparing process and use thereof.

### BACKGROUND

Fibroblast growth factor (FGF) and its tyrosine kinase receptor (FGFR) play important roles in embryonic development, maintenance of homeostasis in various tissues, wound healing processes and metabolic functions. In humans, there are FGFRs (FGFR1-4) and FGFs (FGF 1-14 and FGF 16-23) with a high homology. FGFR contains an extracellular region comprising three immune domains as D1, D2 and D3, a single transmembrane region and a dividing cytoplasmic kinase moiety.

Dysregulation of signaling by FGFR1-4 is associated with several types of cancer. Genomic FGFR mutations including gene amplification, chromosomal translocation and activating mutations induce abnormal activation of the FGF pathway and promote tumor transformation. In particular, the amplification of FGFR3 is associated with the development of solid tumors such as brain cancer, bladder cancer, urothelial cancer, cervical cancer, and intrahepatic cholangiocarcinoma. Additionally, missense FGFR mutations are found in several types of cancer, and FGF-driven signaling and tumor cell proliferation can be enhanced by S249C of FGFR3. The FGFR3 fusion proteins exert a constitutive activation of the kinase domain as cancer driver alterations. The known FGFR3 fusion partners are TACC3, BAIAP2L1, AES, ELAVL3, JAKMIP1, TNIP2, and WHSC1.

Efforts have been made to develop therapeutics targeting FGFR3 to date.

B701 (Vofatamab) is a human immunoglobulin GI monoclonal antibody against FGFR3, and clinical trials have been conducted to confirm whether it exhibits anti-tumor activity and the possibility of combination with docetaxel. When the anti-FGFR3 monoclonal antibody B-701 is administered, it specifically binds and inhibits both wild-type and mutant FGFR3 to inhibit FGFR3 phosphorylation, thereby inhibiting FGFR3 activation and FGFR3-mediated signaling pathways. Thereby, cell proliferation is inhibited, and apoptosis is induced in FGFR3-expressing tumors.

Antibody and ADC targeting FGFR3 entered clinical phases, but further development has been discontinued due to lack of efficacy. There remains a need in the art to develop a new ADC targeting FGFR3.

### SUMMARY

In a first aspect of the present invention, provided is an antibody drug conjugate (ADC) having the structure of formula (I):

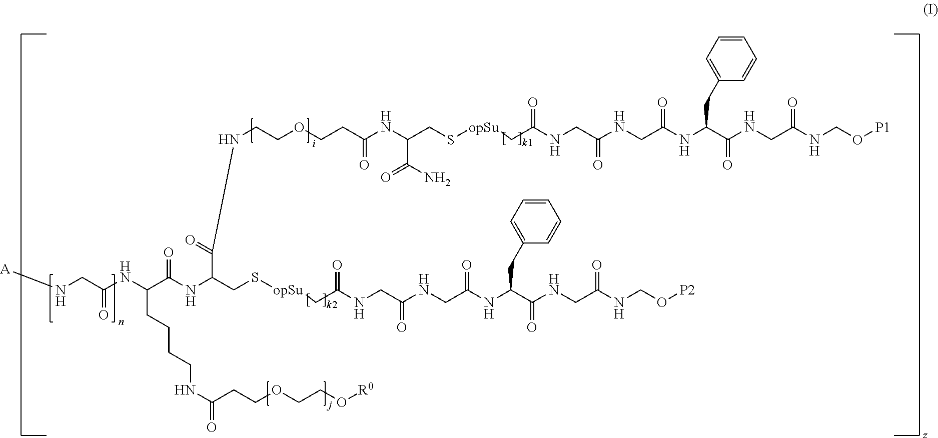

wherein,
A is an anti-FGFR3 antibody or an antigen binding fragment, the antibody or antigen binding fragment is modified to connect with the (Gly)ₙ moiety in the compound of formula (I), wherein the antibody or an antigen binding fragment comprises CDRs: a heavy chain CDR1 comprising amino acid sequence of SEQ ID NO: 1 or having one to three conservative amino acid substitutions compared to SEQ ID NO: 1, a heavy chain CDR2 comprising amino acid sequence of SEQ ID NO: 2 or having one to three conservative amino acid substitutions compared to SEQ ID NO: 2, a heavy chain CDR3 comprising amino acid sequence of SEQ ID NO: 3 or having one to three conservative amino acid substitutions compared to SEQ ID NO: 3, a light chain CDR1 comprising amino acid sequence of SEQ ID NO: 4 or having one to three conservative amino acid substitutions compared to SEQ ID NO: 4, a light chain CDR2 comprising amino acid sequence of SEQ ID NO: 5 or having one to three conservative amino acid substitutions compared to SEQ ID NO: 5, and a light chain CDR3 comprising amino acid sequence of SEQ ID NO: 6 or having one to three conservative amino acid substitutions compared to SEQ ID NO: 6;
z is an integer of 1 to 20; preferably 1 to 4; particularly 2;
opSu is

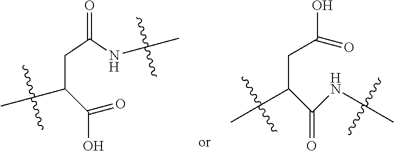 [SMILES: *NC(=O)CC(*)C(=O)O ; *NC(=O)C(*)CC(=O)O]

or a mixture thereof;
R⁰ is C₁₋₁₀ alkyl;
n is any integer of 2 to 20;
k1 and k2 are independently an integer of 1 to 7;
i is an integer of 1-100;
j is an integer of 1-100;
P1 and P2 are independently a payload.

In some embodiments, the connection process between the modified antibody (or antigen binding fragment) and Compound of formula (I) is catalyzed by a ligase.

In some embodiments, the antibody or an antigen binding fragment comprises CDRs: a heavy chain CDR1 comprising amino acid sequence of SEQ ID NO: 1, a heavy chain CDR2 comprising amino acid sequence of SEQ ID NO: 2, a heavy chain CDR3 comprising amino acid sequence of SEQ ID NO: 3, a light chain CDR1 comprising amino acid sequence of SEQ ID NO: 4, a light chain CDR2 comprising amino acid sequence of SEQ ID NO: 5, and a light chain CDR3 comprising amino acid sequence of SEQ ID NO: 6.

In some embodiments, the KD value of the anti-FGFR3 antibody or an antigen binding fragment binding to human FGFR3 and monkey FGFR3 is less than 10 nM.

In some embodiments, the anti-FGFR3 antibody or an antigen binding fragment doesn't bind to mouse FGFR3.

In some embodiments, the payload is a cytotoxin or a fragment thereof, with an optional derivatization in order to connect the payload and linker;
the cytotoxin is selected from the group consisting of taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhy-drovinblastine, dolastatin 10 and analogues, halichondrin B, eribulin, indole-3-oxoacetamide, podophyllotoxins, 7-diethylamino-3-(2′-benzoxazolyl)-coumarin (DBC), discodermolide, laulimalide, camptothecins and derivatives thereof, mitoxantrone, mitoguazone, nitrogen mustards, nitrosoureasm, aziridines, benzodopa, carboquone, meturedepa, uredepa, dynemicin, esperamicin, neocarzinostatin, aclacinomycin, actinomycin, antramycin, bleomycins, actinomycin C, carabicin, carminomycin, cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, tiamiprine, thioguanine, ancitabine, gemcitabine, enocitabine, azacitidine, 6-azauridine, carmofur, cytarabine, dideoxyuridine, doxifluridine, floxuridine, calusterone, dromostanolone propionate, epitiostanol, mepitiostane, testolactone, aminoglutethimide, mitotane, trilostane, flutamide, nilutamide, bicalutamide, leuprorelin acetate, protein kinase inhibitors and a proteasome inhibitors; and/or
selected from vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, pyrrolobenzodiazepine, exatecan and derivatives thereof; and/or
selected from auristatins, especially MMAE, MMAF or MMAD; and/or
selected from exatecan and derivatives thereof, such as DX8951f; and/or
selected from DXd-(1) and DXd-(2); preferably DXd-(1).

In some embodiments, the payload have the structure of formula (II):

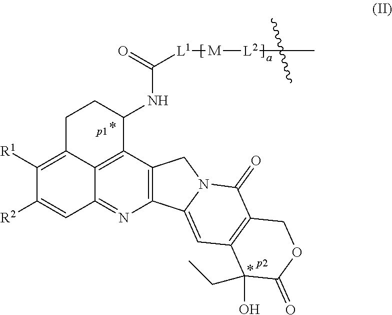

wherein,
a is 0 or 1;
the carbon atoms marked with p1* and p2* each is asymmetric center, and the asymmetric center is S configured, R configured or racemic;
L¹ is selected from C₁₋₆ alkylene, which is unsubstituted or substituted with one substituent selected from halogen, —OH and —NH₂;
M is —CH₂—, —NH— or —O—;
L² is C₁₋₃ alkylene;
R¹ and R² are each independently selected from hydrogen, C₁₋₆ alkyl, halogen and C₁₋₆ alkoxy;

In some embodiments, wherein the payload is selected from:

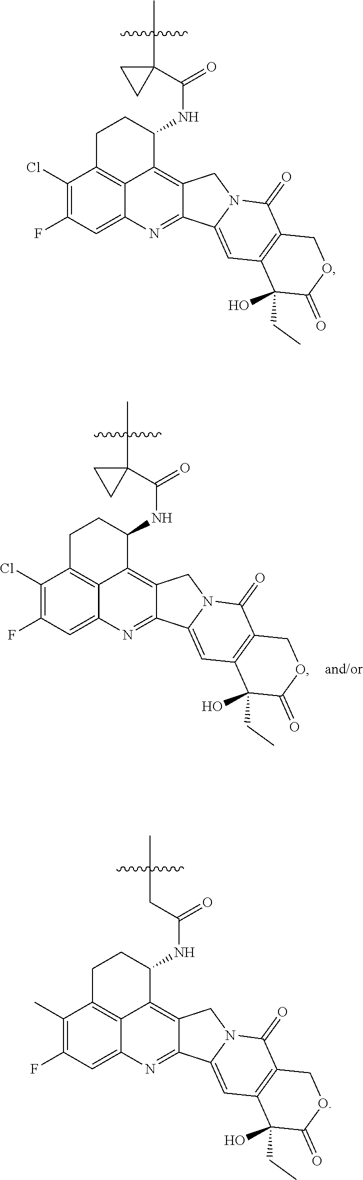 [SMILES: *C1(C(=O)N[C@H]2CCc3c(Cl)c(F)cc4nc5c(c2c34)Cn2c-5cc3c(c2=O)COC(=O)[C@]3(O)CC)CC1 ; *C1(C(=O)N[C@@H]2CCc3c(Cl)c(F)cc4nc5c(c2c34)Cn2c-5cc3c(c2=O)COC(=O)[C@]3(O)CC)CC1 ; *CC(=O)N[C@H]1CCc2c(C)c(F)cc3nc4c(c1c23)Cn1c-4cc2c(c1=O)COC(=O)[C@]2(O)CC]

In some embodiments, the ADC is selected from:

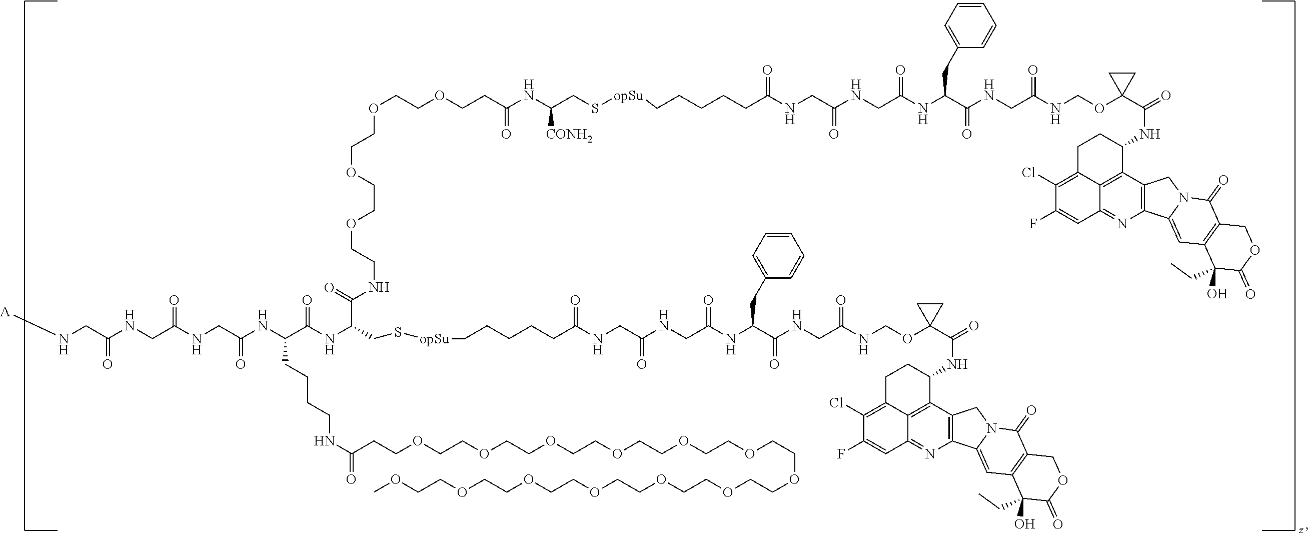

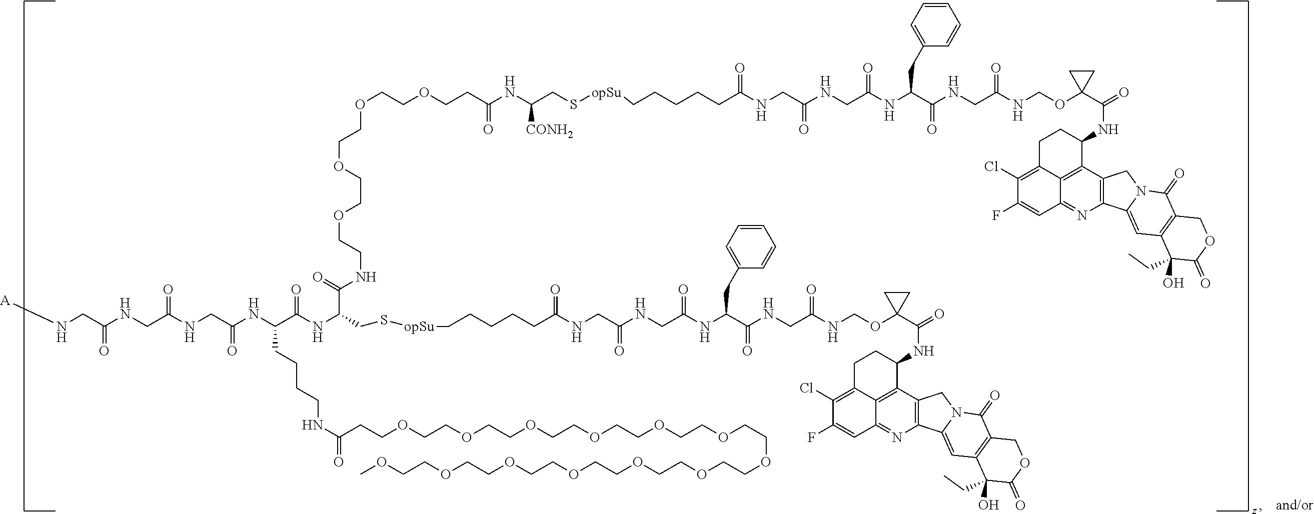

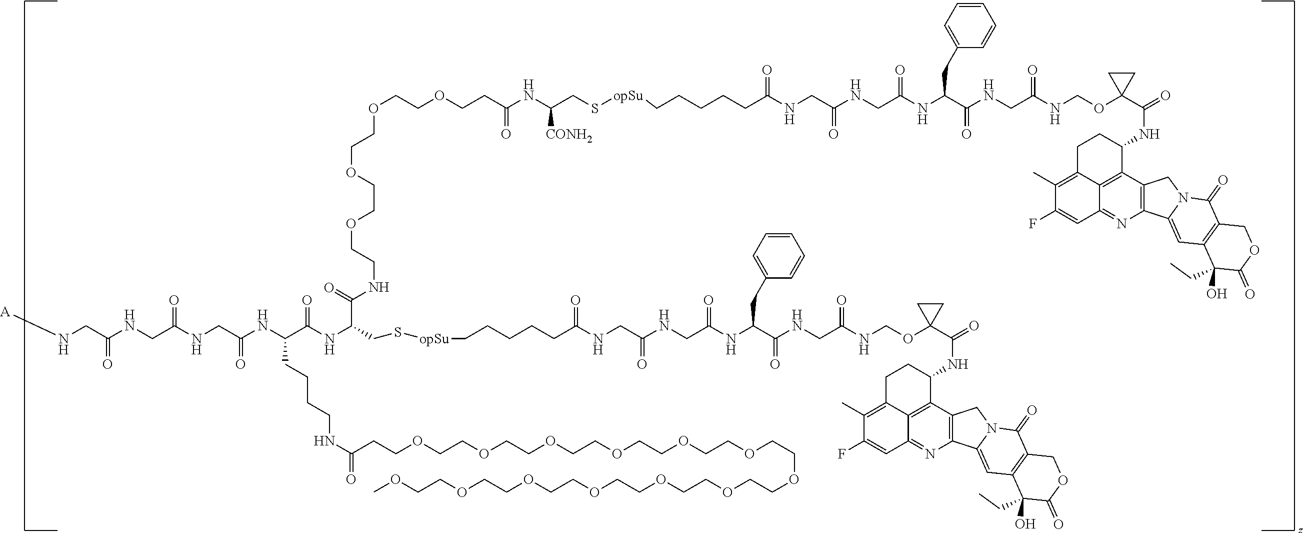

In some embodiments, the antibody or antigen binding fragment comprises a VH domain comprising an amino acid sequence having at least 90% sequence identity to the amino acid sequence of SEQ ID NO: 7; and/or
a VL domain comprising an amino acid sequence having at least 90% sequence identity to the amino acid sequence of SEQ ID NO:8.

In some embodiments, the antibody or antigen binding fragment comprises a VH domain comprising an amino acid sequence of SEQ ID NO: 7; and/or
a VL domain comprising an amino acid sequence of SEQ ID NO: 8.

In some embodiments, the antibody or antigen binding fragment comprises a VH domain comprising an amino acid sequence of SEQ ID NO: 7; and a VL domain comprising an amino acid sequence of SEQ ID NO: 8.

In some embodiments, the antibody or an antigen binding fragment comprises a heavy constant domain comprising an amino acid sequence having at least 90% sequence identity to the amino acid sequence of SEQ ID NO: 9 or SEQ ID NO: 10; and/or
a light constant domain comprising an amino acid sequence having at least 90% sequence identity to the amino acid sequence of SEQ ID NO: 11.

In some embodiments, the antibody or an antigen binding fragment comprises a heavy constant domain comprising an amino acid sequence of SEQ ID NO: 9 or SEQ ID NO: 10; and/or
a light constant domain comprising an amino acid sequence of SEQ ID NO: 11.

In some embodiments, the antibody or an antigen binding fragment comprises a heavy constant domain comprising an amino acid sequence of SEQ ID NO: 9, a light constant domain comprising an amino acid sequence of SEQ ID NO: 11.

In some embodiments, the antibody or an antigen binding fragment comprises a heavy constant domain comprising an amino acid sequence of SEQ ID NO: 10, a light constant domain comprising an amino acid sequence of SEQ ID NO:11.

In some embodiments, wherein the antibody or the antigen-binding fragment comprises C-terminal modification of the heavy chain and/or C-terminal modification of the light chain. In some embodiments, the antibody, Sp and recognition sequence of the ligase donor substrate are sequentially linked; Sp is a spacer sequence selected from GA, GGGGS (SEQ ID NO: 17), GGGGSGGGGS (SEQ ID NO: 18) and GGGGSGGGGSGGGGS (SEQ ID NO:19); the recognition sequence of the ligase donor substrate is LPXTGJ (SEQ ID NO: 20), wherein X can be any single amino acid that is natural or unnatural; J is absent, or is an amino acid fragment comprising 1-10 amino acids.

In some embodiments, the antibody or an antigen binding fragment comprises a heavy chain comprising an amino acid sequence having at least 90% sequence identity to the amino acid sequence of SEQ ID NO: 12 or 13; and/or
a light chain comprising an amino acid sequence having at least 90% sequence identity to the amino acid sequence of SEQ ID NO: 14.

In some embodiments, the antibody or an antigen binding fragment comprises a heavy chain comprising an amino acid sequence of SEQ ID NO: 12, and
a light chain comprising an amino acid sequence of SEQ ID NO: 14.

In some embodiments, the antibody or an antigen binding fragment comprises a heavy chain comprising an amino acid sequence of SEQ ID NO: 13, and
a light chain comprising an amino acid sequence of SEQ ID NO: 14.

In some embodiments, the antibody drug conjugate has a drug to antibody ratio (DAR) of an integer or non-integer of 1 to 20, particularly 2 to 8. In some embodiments, the value of DAR is about 1, about 2, about 3, about 4, about 5, about 6, about 7, about 8, about 9, about 10, about 11, about 12, about 13, about 14, about 15, about 16, about 17, about 18, about 19, about 20, or the range between any two values (including the end value).

In a second aspect, provided is a pharmaceutical composition comprising the ADC of the present disclosure, and at least one pharmaceutically acceptable carrier.

In some embodiments, the pharmaceutical composition comprises alkylating agents. In some embodiments, wherein the alkylating agents comprises temozolomide or derivates thereof.

In a third aspect, provided is a pharmaceutical combination comprising the ADC of the present disclosure and alkylating agents.

In some embodiments, the pharmaceutical combination comprises the ADC of the present disclosure and temozolomide or derivates thereof. In some embodiments, the pharmaceutical combination comprises the ADC and temozolomide. In some embodiments, the pharmaceutical combination further comprises pharmaceutically acceptable carrier.

In a fourth aspect, provided is a kit comprising the ADC of the present disclosure, the pharmaceutical composition or pharmaceutical combination.

In some embodiments, the kit comprises a first packaging unit, comprising the conjugate having the structure of formula (I),
a second packaging unit, comprising the alkylating agents; and
optionally an instruction for administrating the conjugate and alkylating agents to a subject.

In some embodiments, the subject suffers a disease. In some embodiments, the disease is FGFR3-mediated disease. In some embodiments, the alkylating agents is temozolomide or derivates thereof.

In some embodiments, the disease is a tumor. In some embodiments, the disease includes FGFR3-positive tumor. In some embodiments, the disease includes tumor overexpressing FGFR3 or tumor with FGFR3 alteration. In some embodiments, the disease includes tumor with FGFR3 infusion (such as TACC3 fusion, intracellular fusion) or tumor with FGFR3 gene mutation (such as Y373C, G380R, S371C, S249C or R248C). In some embodiments, the disease is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendothelial sarcoma, synovioma, mesothelioma, Ewing's tumor, leiomyosarcoma, rhabdomyosarcoma, colon cancer, pancreatic cancer, breast cancer, thyroid cancer, endometrial cancer, melanoma, ovarian cancer, prostate cancer, squamous cell carcinoma, basal cell carcinoma, adenocarcinoma, sweat gland carcinoma, sebaceous carcinoma, papillary carcinoma, papillary adenocarcinoma, cystadenocarcinoma, medullary carcinoma, bronchial carcinoma, renal cell carcinoma, liver cancer, bile duct cancer, choriocarcinoma, seminoma, embryonal carcinoma, Wilms' tumor, cervical cancer, testicular tumor, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, neuroblastoma and retinoblastoma, multiple myeloma. In some embodiments, the disease is selected from: brain cancer, bladder cancer, urothelial cancer, cervical cancer, or intrahepatic cholangiocarcinoma. In some embodiments, the disease is glioblastoma, bladder cancer, or multiple myeloma.

In a fifth aspect, provided is use of the ADC of the present disclosure, pharmaceutical combination or the pharmaceutical composition of the present disclosure in the manufacture of a medicament for treating a disease; wherein the disease is FGFR3-mediated disease.

In some embodiments, the disease is a tumor. In some embodiments, the disease includes FGFR3-positive tumor. In some embodiments, the disease includes tumor overexpressing FGFR3 or tumor with FGFR3 alteration. In some embodiments, the disease includes tumor with FGFR3 infusion (such as TACC3 fusion, intracellular fusion) or tumor with FGFR3 gene mutation (such as Y373C, G380R, S371C, S249C or R248C). In some embodiments, the disease is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendothelial sarcoma, synovioma, mesothelioma, Ewing's tumor, leiomyosarcoma, rhabdomyosarcoma, colon cancer, pancreatic cancer, breast cancer, thyroid cancer, endometrial cancer, melanoma, ovarian cancer, prostate cancer, squamous cell carcinoma, basal cell carcinoma, adenocarcinoma, sweat gland carcinoma, sebaceous carcinoma, papillary carcinoma, papillary adenocarcinoma, cystadenocarcinoma, medullary carcinoma, bronchial carcinoma, renal cell carcinoma, liver cancer, bile duct cancer, choriocarcinoma, seminoma, embryonal carcinoma, Wilms' tumor, cervical cancer, testicular tumor, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, neuroblastoma and retinoblastoma, multiple myeloma. In some embodiments, the disease is selected from: brain cancer, bladder cancer, urothelial cancer, cervical cancer, or intrahepatic cholangiocarcinoma. In some embodiments, the disease is glioblastoma, bladder cancer, or multiple myeloma.

In a sixth aspect, provided is a method for treating a subject suffering from a disease or reducing the likelihood of disease progression, comprises administering the conjugate, the pharmaceutical combination, the pharmaceutical composition, or the kit, wherein the disease is FGFR3-mediated disease.

In some embodiments, the disease is a tumor. In some embodiments, the disease includes FGFR3-positive tumor. In some embodiments, the disease includes tumor overexpressing FGFR3 or tumor with FGFR3 alteration. In some embodiments, the disease includes tumor with FGFR3 infusion (such as TACC3 fusion, intracellular fusion) or tumor with FGFR3 gene mutation (such as Y373C, G380R, S371C, S249C or R248C). In some embodiments, the disease is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendothelial sarcoma, synovioma, mesothelioma, Ewing's tumor, leiomyosarcoma, rhabdomyosarcoma, colon cancer, pancreatic cancer, breast cancer, thyroid cancer, endometrial cancer, melanoma, ovarian cancer, prostate cancer, squamous cell carcinoma, basal cell carcinoma, adenocarcinoma, sweat gland carcinoma, sebaceous carcinoma, papillary carcinoma, papillary adenocarcinoma, cystadenocarcinoma, medullary carcinoma, bronchial carcinoma, renal cell carcinoma, liver cancer, bile duct cancer, choriocarcinoma, seminoma, embryonal carcinoma, Wilms' tumor, cervical cancer, testicular tumor, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, neuroblastoma and retinoblastoma, multiple myeloma. In some embodiments, the disease is selected from: brain cancer, bladder cancer, urothelial cancer, cervical cancer, or intrahepatic cholangiocarcinoma. In some embodiments, the disease is glioblastoma, bladder cancer, or multiple myeloma.

In some embodiments, the conjugate and alkylating agents are administered simultaneously as part of the same pharmaceutical formulation. In some embodiments, the conjugate and alkylating agents are administered simultaneously as part of the different pharmaceutical formulation. In some embodiments, the alkylating agents is temozolomide or derivates thereof.

In some embodiments, the conjugate and alkylating agents are administered at different times.

In another aspect, provided is use of an effective amount of the conjugate for the manufacture of a medicament for the treatment of a subject with cancer to be used in combination with an effective amount of the alkylating agents.

In some embodiments, the conjugate and alkylating agents are administered simultaneously as part of the same pharmaceutical formulation. In some embodiments, the conjugate and alkylating agents are administered simultaneously as part of the different pharmaceutical formulation. In some embodiments, the alkylating agents is temozolomide or derivates thereof.

In some embodiments, the conjugate and alkylating agents are administered at different times.

In some embodiments, the disease includes FGFR3-positive tumor. In some embodiments, the disease includes tumor overexpressing FGFR3 or tumor with FGFR3 alteration. In some embodiments, the disease includes tumor with FGFR3 infusion (such as TACC3 fusion, intracellular fusion) or tumor with FGFR3 gene mutation (such as Y373C, G380R, S371C, S249C or R248C). In some embodiments, the disease is selected from the group consisting of: fibrosarcoma, myxosarcoma, liposarcoma, chondrosarcoma, osteogenic sarcoma, chordoma, angiosarcoma, endotheliosarcoma, lymphangiosarcoma, lymphangioendothelial sarcoma, synovioma, mesothelioma, Ewing's tumor, leiomyosarcoma, rhabdomyosarcoma, colon cancer, pancreatic cancer, breast cancer, thyroid cancer, endometrial cancer, melanoma, ovarian cancer, prostate cancer, squamous cell carcinoma, basal cell carcinoma, adenocarcinoma, sweat gland carcinoma, sebaceous carcinoma, papillary carcinoma, papillary adenocarcinoma, cystadenocarcinoma, medullary carcinoma, bronchial carcinoma, renal cell carcinoma, liver cancer, bile duct cancer, choriocarcinoma, seminoma, embryonal carcinoma, Wilms' tumor, cervical cancer, testicular tumor, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, neuroblastoma and retinoblastoma, multiple myeloma. In some embodiments, the disease is selected from: brain cancer, bladder cancer, urothelial cancer, cervical cancer, or intrahepatic cholangiocarcinoma. In some embodiments, the disease is glioblastoma, bladder cancer, or multiple myeloma.

### BRIEF DESCRIPTION OF THE DRAWINGS

FIG. 1 shows the SDS-PAGE results for the antibody prepared in Example 3.

FIG. 2 shows the specificity of A9 to FGFR3 in the affinity ELISA analysis.

FIG. 3 shows the comparison of specificities between A9 and A9Q in the affinity to FGFR3 in the affinity ELISA analysis.

FIG. 4 shows that the comparison of specificities between A9 and ADC19 in the affinity to FGFR3 in the affinity ELISA analysis.

FIG. 5 shows that the specificity of A9 to FGFR3 positive cells in the cell binding analysis.

FIG. 6.1 shows that the comparison of specificities between A9 and A9Q in the affinity to FGFR3 positive cells in the cell binding analysis, FIG. 6.2 shows cell binding analysis of ADC20.

FIG. 7.1 and FIG. 7.2 show in vivo efficacy test in glioblastoma PDX models.

FIGS. 8.1-8.4 show in vivo efficacy test in bladder cancer CDX models.

FIG. 9 shows in vivo efficacy test in multiple myeloma CDX models.

FIG. 10 shows internalization activity of ADC20.

FIG. 11 shows bystander killing effect of ADC19 and ADC20.

FIG. 12.1 and FIG. 12.2 show in vivo efficacy test in glioblastoma PDX models.

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patent applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “i is an integer of 1 to 20” means that i is any integer of 1 to 20, for example, i can be 1, 2, 3, 4, 5, 6, 7, 8, 9, 10, 11, 12, 13, 14, 15, 16, 17, 18, 19 or 20.

Other similar expressions such as j, k1, k2, n, k and z should also be understood in a similar manner. “Conservative amino acid substitution” refers to the replacement of one amino acid residue with another amino acid residue having a side chain (R group) of similar chemical nature (e.g., charge or hydrophobicity). Generally, conservative amino acid substitutions do not substantially alter the functional properties of the protein. Examples of amino acid classes with a side chain of similar chemical nature include: 1) an aliphatic side chain: glycine, alanine, valine, leucine and isoleucine; 2) an aliphatic hydroxyl side chain: serine and threonine; 3) an amide-containing side chain: asparagine and glutamine; 4) an aromatic side chain: phenylalanine, tyrosine and tryptophan; 5) a basic side chain: lysine, arginine and histidine; and 6) an acidic side chain: aspartic acid and glutamic acid.

Unless the context clearly dictates otherwise, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within 10% of a specified value, or a wider range.

The term “optional” or “optionally” means the event described subsequent thereto may, but not necessarily happen, and the description includes the cases wherein said event or circumstance happens or does not happen.

The expressions “comprising”, “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

As used herein, the term “antibody” is used in a broad way and particularly includes intact monoclonal antibodies, polyclonal antibodies, monospecific antibodies, multispecific antibodies (e.g., bispecific antibodies), and antibody fragments, as long as they have the desired biological activity. The antibody may be of any subtype (such as IgG, IgE, IgM, IgD, and IgA) or subclass, and may be derived from any suitable species. In some embodiments, the antibody is of human or murine origin. The antibody may also be a fully human antibody, humanized antibody or chimeric antibody prepared by recombinant methods.

Monoclonal antibodies are used herein to refer to antibodies obtained from a substantially homogeneous population of antibodies, i.e., the individual antibodies constituting the population are identical except for a small number of possible natural mutations.

Monoclonal antibodies are highly specific for a single antigenic site. The word “monoclonal” refers to that the characteristics of the antibody are derived from a substantially homogeneous population of antibodies and are not to be construed as requiring some particular methods to produce the antibody.

An intact antibody or full-length antibody essentially comprises the antigen-binding variable region(s) as well as the light chain constant region(s) (CL) and heavy chain constant region(s) (CH), which could include CH₁, CH₂, CH₃ and CH₄, depending on the subtype of the antibody. An antigen-biding variable region (also known as a fragment variable region, Fv fragment) typically comprises a light chain variable region (VL) and a heavy chain variable region (VH). A constant region can be a constant region with a native sequence (such as a constant region with a human native sequence) or an amino acid sequence variant thereof. The variable region recognizes and interacts with the target antigen. The constant region can be recognized by and interacts with the immune system.

An antibody fragment may comprise a portion of an intact antibody, preferably its antigen binding region or variable region. Examples of antibody fragments include Fab, Fab′, F(ab′)₂, Fd fragment consisting of VH and CH₁ domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a VL and a CL) and another chain, wherein the said other chain comprises a variable domain of the heavy chain (VH) and a constant region domain of the heavy chain (CH₁). The F(ab′)₂ fragment is an antibody fragment obtained by pepsin digestion of an immunoglobulin at pH 4.0-4.5, or a fragment having the same structure produced by, for example, recombinant expression. The F(ab′)₂ fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)₂ fragment (one heavy chain and one light chain). The antibody fragment may comprise a plurality of chains joined together, for example, via a disulfide bond and/or via a peptide linker. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at least or about 200 amino acids. An antigen-binding fragment can include any antibody fragment that, when inserted into an antibody framework (e.g., by substitution of the corresponding region), can result in an antibody that immunospecifically binds to the antigen.

Antibodies according to the present disclosure can be prepared using techniques well known in the art, such as the following techniques or a combination thereof: recombinant techniques, phage display techniques, synthetic techniques, or other techniques known in the art. For example, a genetically engineered recombinant antibody (or antibody mimic) can be expressed by a suitable culture system (e.g., E. coli or mammalian cells). The engineering can refer to, for example, the introduction of a ligase-specific recognition sequence at its terminals.

As used herein, the term “antibody-drug conjugate” is referred to as “conjugate”.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

A spacer is a structure that is located between different structural modules and can spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid, a sequence containing a plurality of amino acids, for example, a sequence containing two amino acids such as GA, etc., or, for example, GGGGS (SEQ ID NO: 17), GGGGSGGGGS (SEQ ID NO: 18), GGGGSGGGGSGGGGS (SEQ ID NO: 19), etc. Self-immolative spacers are covalent assemblies tailored to correlate the cleavage of two chemical bonds after activation of a protective part in a precursor: Upon stimulation, the protective moiety (such as a cleavable sequence) is removed, which generates a cascade of disassembling reactions leading to the temporally sequential release of smaller molecules. Examples of self-immolative spacers include but not limited to PABC (p-benzyloxycarbonyl), acetal, heteroacetal and the combination thereof.

The term “alkyl” refers to a saturated aliphatic hydrocarbon group consisting of carbon atoms and hydrogen atoms, which is connected to the rest of the molecule through a single bond. Alkyl groups comprise straight alkyl, branched alkyl or cyclic alkyl (cycloalkyl) or partially cyclic alkyl (e.g., cycloalkyl-linear alkyl and cycloalkyl-branched alkyl). The alkyl group may contain 1 to 10 carbon atoms, referring to C₁₋₁₀ alkyl group, for example, C₁₋₆ alkyl group, C₁₋₄ alkyl group, C₁₋₃ alkyl group, C₁₋₂ alkyl, C₃ alkyl, C₄ alkyl, C₃₋₆ alkyl. Non-limiting examples of linear alkyl groups include but are not limited to methyl, ethyl, n-propyl, n-butyl, n-pentyl, n-hexyl, etc. Non-limiting examples of branched alkyl groups include but are not limited to isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, etc.

The terms “cyclic alkyl” and “cycloalkyl” have the same meaning and are used herein interchangeably. Cyclic alkyl groups can include mono- or polycyclic (e.g., having 2 or more than 2 fused rings) groups. In a multicyclic cycloalkyl, two or more rings can be fused or bridged or spiro together. Ring-forming carbon atoms of a cyclic alkyl group can be optionally substituted by oxo (i.e., C(O)). Cyclic alkyl groups can have 3, 4, 5, 6, 7, 8, 9, or 10 ring-forming carbon atoms (C₃₋₁₀). Examples of cyclic alkyl groups include but are not limited to cyclopropyl, cyclobutyl, cyclopentyl, cyclohexyl, bicyclo[1.1.1]pentyl and bicyclo[2.1.1]hexyl. In some embodiments, the cyclic alkyl is a C₃₋₆ monocyclic or bicyclic cyclic alkyl, preferably C₃₋₆ monocyclic cyclic alkyl, especially cyclopropyl.

The term “partially cyclic” refers to that the group contains one or more cyclic moieties and one or more acyclic (i.e. linear or branched) moieties. Partially cyclic alkyl groups can include cyclic alkyl-linear alkyl groups and cyclic alkyl-branched alkyl groups. Partially cyclic alkyl groups can have 4, 5, 6, 7, 8, 9, or 10 carbon atoms (C₃₋₁₀), including ring-forming carbon atoms and non-ring-forming carbon atoms. Examples of partially cyclic alkyl groups include but are not limited to C₃₋₉ cyclic alkyl-C₁ alkyl groups, C₃₋₈ cyclic alkyl-C₂ alkyl groups, C₃₋₇ cyclic alkyl-C₃ linear alkyl groups, C₃₋₆ cyclic alkyl-C₄ linear alkyl groups, C₃₋₅ cyclic alkyl-C₅ linear alkyl groups, C₃₋₄ cyclic alkyl-C₆ linear alkyl groups, C₃ cyclic alkyl-C₇ linear alkyl groups, C₃₋₇ cyclic alkyl-C₃ branched alkyl groups, C₃₋₆ cyclic alkyl-C₄ branched alkyl groups, C₃₋₅ cyclic alkyl-C₅ branched alkyl groups, C₃₋₄ cyclic alkyl-C₆ branched alkyl groups, and C₃ cyclic alkyl-C₇ branched alkyl groups. In some embodiments, the partially cyclic alkyl is a C₃₋₉ cyclic alkyl-C₁ alkyl group, preferably a C₃₋₆ cyclic alkyl-C₁₋₂ alkyl group, C₃₋₆ cyclic alkyl-C₁ alkyl group, more preferably C₃₋₄ cyclic alkyl-C₁₋₂ alkyl group, particularly C₃₋₄ cyclic alkyl-C₁ alkyl group, especially cyclopropyl-methyl.

A bivalent radical refers to a group obtained from the corresponding monovalent radical by removing one hydrogen atom from a carbon atom with free valence electron(s). A bivalent radical have two connecting sites which are connected to the rest of the molecule, wherein the two connecting sites may be on the same atom or on two different atoms of the bivalent radical.

An “alkylene” or an “alkylidene” refers to a saturated divalent hydrocarbon group.

Alkylene groups comprise linear, branched, cyclic or partially cyclic groups. Examples of linear alkylene groups include but are not limited to methylene (—CH₂—), —(CH₂)₂—, —(CH₂)₃—, —(CH₂)₄—, —(CH₂)₅—, —(CH₂)₆—, etc. Examples of branched alkylene groups include but are not limited to —CH(CH₃)—, —CH(C₂H₅)—, —CH(CH₃)—CH₂—, —CH(C₃H)—, —CH(C₂H₅)—CH₂—, —C(CH₃)₂—CH₂—, —(CH(CH₃))₂—, —CH(CH₃)—(CH₂)₂—, —CH₂—CH(CH₃)—CH₂—, —CH(C₄H₉)—, —C(CH₃)(C₃H)—, —C(C₂H₅)₂—, —CH(C₃H)—CH₂—, —CH(C₂H₅)—CH(CH₃)—, —CH(C₂H₅)—(CH₂)₂—, —CH₂—CH(C₂H₅)—CH₂—, —C(CH₃)₂—(CH₂)₂—, —CH₂—C(CH₃)₂—CH₂—, —CH(CH₃)—(CH₂)₃—, —CH₂—CH(CH₃)—(CH₂)₂—, —CH(C₅H₁₁)—, —C(C₂H₅)(C₃H₇)—, —C(CH₃)(C₄H₉)—, —CH(C₄H₉)—CH₂—, —C(C₂H₅)₂—CH₂—, —C(CH₃)(C₃H)—CH₂—, —CH(C₂H₅)—CH(C₂H₅)—, —CH(CH₃)—CH(C₃H)—, —C(CH₃)₂—C(CH₃)₂—, —CH(C₃H₇)—(CH₂)₂—, —CH₂—CH(C₃H₇)—CH₂—, —CH(C₂H₅)—C(CH₃)₂—, —C(CH₃)₂—CH(CH₃)—CH₂—, —CH(CH₃)—C(CH₃)₂—CH₂—, —CH(C₂H₅)—CH(CH₃)—CH₂—, —CH(CH₃)—CH(C₂H₅)—CH₂—, —CH(CH₃)—CH₂—CH(C₂H₅)—, —CH(CH₃)—C(CH₃)₂—CH₂—, —(CH(CH₃))₃—, —C(CH₃)₂—(CH₂)₃—, —CH(C₂H₅)—(CH₂)₃—, —CH₂—CH(C₂H₅)—(CH₂)₂—, —CH₂—CH(CH₃)—CH(CH₃)—CH₂—, —(CH(CH₃))₂—(CH₂)₂—, —CH(CH₃)—(CH₂)₂—CH(CH₃)—, —(CH₂)₂—CH(CH₃)—(CH₂)₂—, —CH₂—CH(CH₃)—(CH₂)₃—, —CH(CH₃)—(CH₂)₄—, etc.

The terms “cyclic alkylene” and “cycloalkylene” have the same meaning and are used herein interchangeably. Examples of cyclic alkylene groups include but are not limited to cyclopropylene, cyclobutylene, cyclopentylene, cyclohexylene, cycloheptylene and cyclooctylene, and divalent multicyclic alkyl groups containing fused, spiro or bridged rings. In some embodiments, the cyclic alkylene is a C₃₋₆ cyclic alkylene group, particularly C₃₋₄ cyclic alkylene group, especially cyclopropyl.

Partially cyclic alkylene groups can include bivalent radicals wherein the two connecting sites which are connected to the rest of the molecule can be both on the one or more linear or branched alkyl moieties, or both on the one or more cyclic alkyl moieties, or respectively on a cyclic alkyl moiety and a linear or branched alkyl moiety. Examples of partially cyclic alkylene groups include but are not limited to: (1) cyclopropylene, cyclobutylene, cyclopentylene, cyclohexylene, cycloheptylene and cyclooctylene, and divalent multicyclic alkyl groups containing fused, spiro or bridged rings, which are each independently substituted by one or more linear or branched alkyl groups; (2) linear or branched alkylene groups, which are each independently substituted by one or more cyclic alkyl groups; and (3) a group formed by combining one or more cyclic alkylene groups and one or more linear or branched alkylene groups, provided that a chemically stable structure is formed. In some embodiments, the partially cyclic alkylene is a C₃₋₉ cyclic alkyl-C₁ alkylene group, preferably a C₃₋₆ cyclic alkyl-C₁₋₂ alkylene group, C₃₋₆ cyclic alkyl-C₁ alkylene group, more preferably C₃₋₄ cyclic alkyl-C₁₋₂ alkylene group, particularly C₃₋₄ cyclic alkyl-C₁ alkylene group, especially cyclopropyl-methylene.

As used herein, the expressions “antibody-conjugated drug”, “ADC” and “antibody-drug conjugate” has the same meaning.

### Compound of Formula (III)

In some embodiments, the linker-payload intermediate of the ADC has the structure of formula (III):

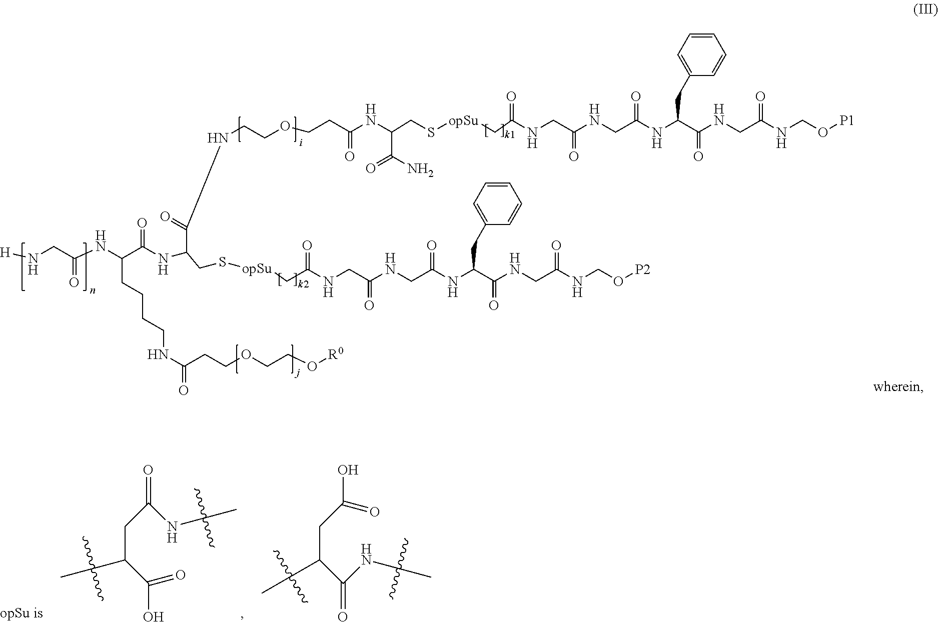

or a mixture thereof;
R⁰ is C₁₋₁₀ alkyl;
n is any integer of 2 to 20;
k1 and k2 are independently an integer of 1 to 7;
i is an integer of 1-100;
j is an integer of 1-100;
P1 and P2 are independently a payload having the structure of formula (II).

In an embodiment, R⁰ is C₁₋₆ alkyl. In a preferred embodiment, R⁰ is C₁₋₃ alkyl. In a particular embodiment, R⁰ is methyl.

In an embodiment, n is an integer of 2 to 5. In a particular embodiment, n is 3.

In an embodiment, k1 and k2 are independently 1, or 3 or 5. In a particular embodiment, k1 and k2 are independently 5.

In an embodiment, i is independently an integer of 1 to 20, preferably 1 to 12, more preferably 2 to 8. In a particular embodiment, i is 4.

In an embodiment, j is independently an integer of 1 to 20, preferably 1 to 12, more preferably 8 to 12, especially 8 or 12. In a particular embodiment, j is 12.

In an embodiment, the compound of formula (III) has the structure of formula (III-1)

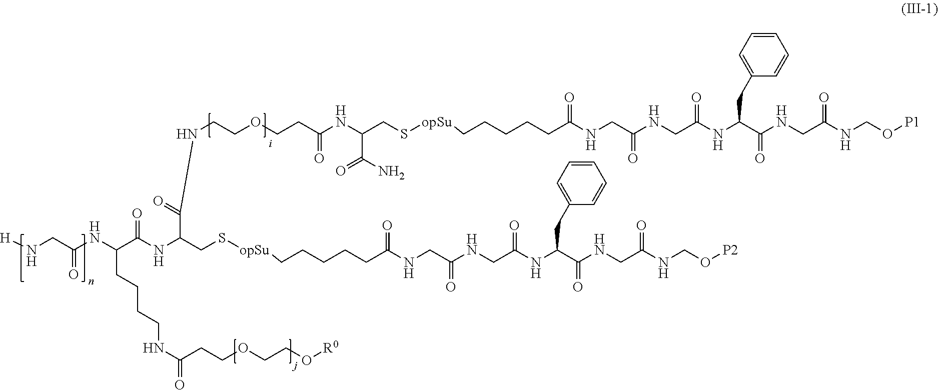

wherein,
P1, P2, R⁰, opSu, n, i and j are as defined in formula (III).

In an embodiment, the compound of formula (III) is selected from the group consisting of:

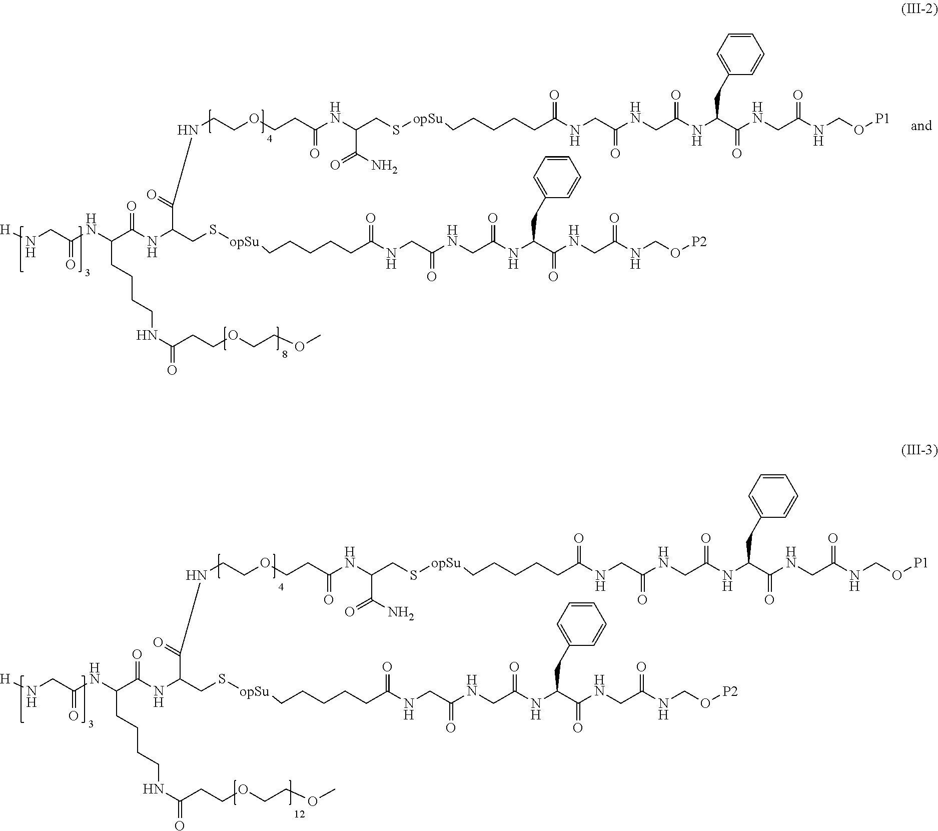

### Preparation of the Compound of Formula (III)

In an embodiment, compound of formula (III) can be synthesized by connecting a linker with a payload or by connecting a serial of suitable building blocks. Such building blocks can be easily designed by retrosynthetic analysis, and any reaction known in the art can be used.

In an embodiment, provided is a compound having the structure of formula (IV):

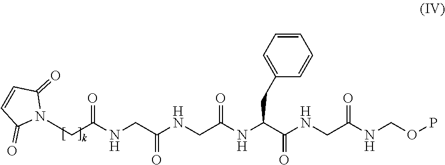

wherein,
k is an integer of 1 to 7;
P is a payload having the structure of formula (II), wherein the structure of formula (II) is as defined above.

In some embodiments, k is about 1, about 2, about 3, about 4, about 5, about 6, or about 7. In an embodiment, k is 1, or 3 or 5. In a particular embodiment, k is 5.

The compound of formula (III) can be synthesized using the compound of formula (II)/(IV) and other necessary building blocks, using a method similar to the synthetic method as disclosed in EP2907824A (e.g., synthetic method for formula (2) or (2b) of EP2907824A). Suitable building blocks include but not limited to Linker-payload intermediate 2 and Linker-payload intermediate 1.

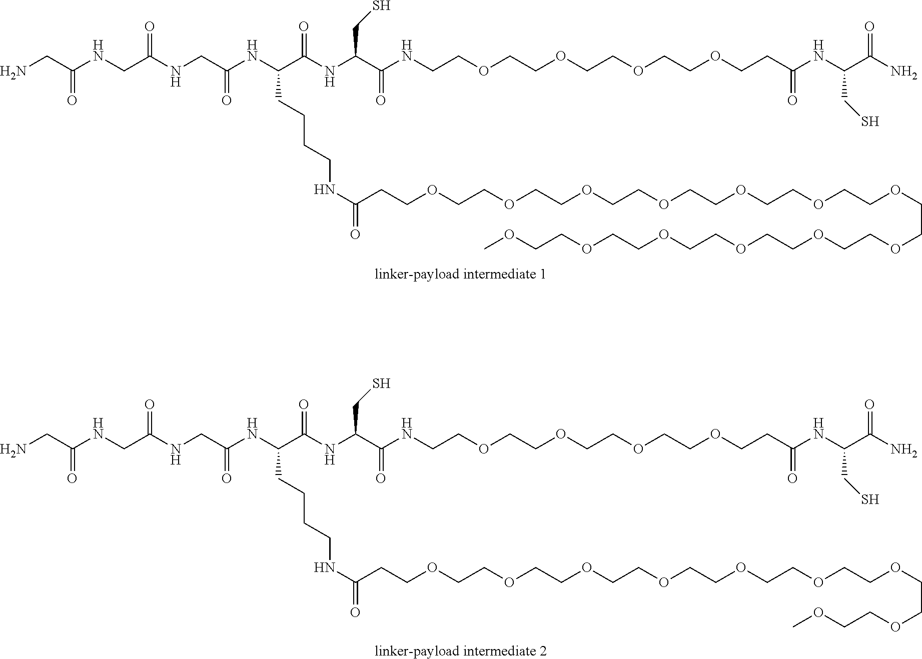

Then the maleimide group of formula (IV) therein can be reacted to a thiol group on another building block. The resulting thiosuccinimide is unstable under physiological conditions and is liable to reverse Michael addition which leads to cleavage at the connection site. Moreover, when another thiol compound is present in the system, thiosuccinimide may also undergo thiol exchange with the other thiol compound. Both of these reactions cause the fall-off of the payload and result in toxic side effects. The thiosuccinimide is then subjected to ring opening reaction. The compound of formula (III) can then be obtained.

Method of ring opening reaction can be found in WO2015165413A1. The compound comprising ring-opened succinimide moiety can be purified by semi-preparative/preparative HPLC or other suitable separation means to obtain with high purity and defined composition, regardless of the efficiency of the succinimide ring opening reaction.

In the present disclosure, when applied in the linker-payload (linker-small molecule intermediate), the ring-opened succinimide structure no longer undergoes reverse Michael addition or thiol exchange, and thus the product is more stable.

### Moiety Comprising Recognition Sequence of the Ligase Acceptor and Donor Substrate

In an embodiment, the (Gly)ₙ moiety of the compound of formula (III) is a recognition sequence of a ligase donor substrate, which facilitates enzyme-catalyzed coupling of compound of formula (III) with the an antibody or an antigen binding fragment under the catalysis of the ligase. The antibody or the antigen binding fragment is optionally modified and comprises the corresponding recognition sequence of a ligase acceptor substrate.

In an embodiment, the ligase is a transpeptidase. In an embodiment, the ligase is selected from the group consisting of a natural transpeptidase, an unnatural transpeptidase, variants thereof, and the combination thereof. Unnatural transpeptidase enzymes can be, but are not limited to, those obtained by engineering of natural transpeptidase. In a preferred embodiment, the ligase is selected from the group consisting of a natural Sortase, an unnatural Sortase, and the combination thereof. The species of natural Sortase include Sortase A, Sortase B, Sortase C, Sortase D, Sortase L. plantarum, etc. (US20110321183A1). The type of ligase corresponds to the ligase recognition sequence and is thereby used to achieve specific conjugation between different molecules or structural fragments.

In an embodiment, the (Gly)ₙ moiety of the compound of formula (III) is a recognition sequence of a ligase acceptor substrate; and the antibody or the antigen binding fragment is optionally modified and comprises the corresponding recognition sequence of a ligase donor substrate.

In some embodiments, the ligase is a Sortase selected from Sortase A, Sortase B, Sortase C, Sortase D and Sortase L. plantarum.

In a particular embodiment, the ligase is Sortase A from Staphylococcus aureus. Accordingly, the ligase recognition sequence of the ligase donor substrate may be the typical recognition sequence LPXTG (SEQ ID NO: 21) of the enzyme, wherein X can be any single amino acid that is natural or unnatural. In yet another particular embodiment, the recognition sequence of the ligase donor substrate is LPXTGJ (SEQ ID NO: 20), wherein X can be any single amino acid that is natural or unnatural; J is absent, or is an amino acid fragment comprising 1-10 amino acids, optionally labeled. In an embodiment, J is absent. In yet another embodiment, J is an amino acid fragment comprising 1-10 amino acids, wherein each amino acid is independently any natural or unnatural amino acid. In another embodiment, J is (Gly)ₘ, wherein m is an integer of 1 to 10. In yet another particular embodiment, the recognition sequence of the ligase donor substrate is LPETG (SEQ ID NO: 28). In another particular embodiment, the recognition sequence of the ligase donor substrate is LPETGG (SEQ ID NO: 22).

In an embodiment, the ligase is Sortase B from Staphylococcus aureus and the corresponding donor substrate recognition sequence can be NPQTN (SEQ ID NO: 23). In another embodiment, the ligase is Sortase B from Bacillus anthracis and the corresponding donor substrate recognition sequence can be NPKTG (SEQ ID NO: 24).

In yet another embodiment, the ligase is Sortase A from Streptococcus pyogenes and the corresponding donor substrate recognition sequence can be LPXTGJ, wherein J is as defined above. In another embodiment, the ligase is Sortase subfamily 5 from Streptomyces coelicolor, and the corresponding donor substrate recognition sequence can be LAXTG (SEQ ID NO: 25).

In yet another embodiment, the ligase is Sortase A from Lactobacillus plantarum and the corresponding donor substrate recognition sequence can be LPQTSEQ (SEQ ID NO: 26).

The ligase recognition sequence can also be other totally new recognition sequence for transpeptidase optimized by manual screening.

### Conjugates and Preparation Thereof

Furthermore, the payload-bearing compound (compound of formula (III)) which has the moiety comprising ligase recognition sequence can be conjugated with anti-FGFR3 or an antigen binding fragment comprising a ligase recognition sequence.

In yet another aspect provided is a conjugate having the structure of formula (I):

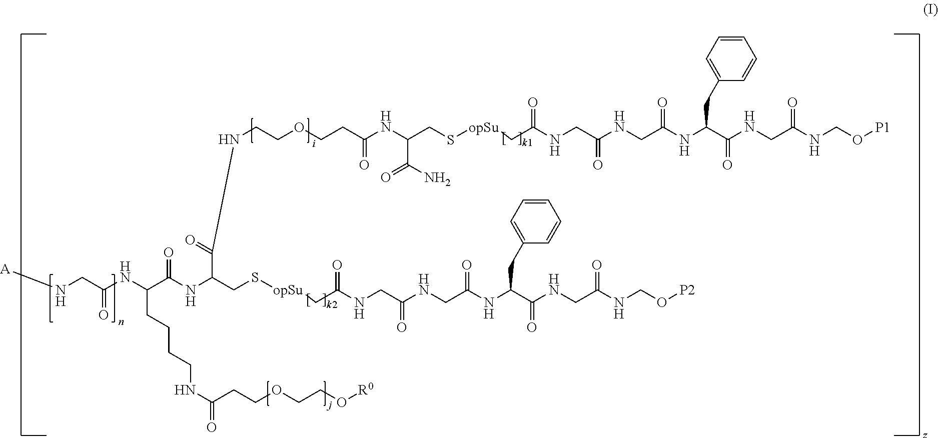

wherein,
A is an anti-FGFR3 antibody or an antigen binding fragment thereof;
z is an integer of 1 to 20;
P1, P2, R⁰, opSu, n, k1, k2, i and j are as defined as above.

In an embodiment, the antibody or antigen binding fragment is modified to connect with the (Gly)ₙ moiety in the compound of formula (III).

In an embodiment, z is about 1 to 20. In some embodiments, z is about 1, about 2, about 3, about 4, about 5, about 6, about 7, about 8, about 9, about 10, about 11, about 12, about 13, about 14, about 15, about 16, about 17, about 18, about 19, about 20, or the range between any two values (including the end value).

In an embodiment, the compound of formula (I) is selected from the group consisting of

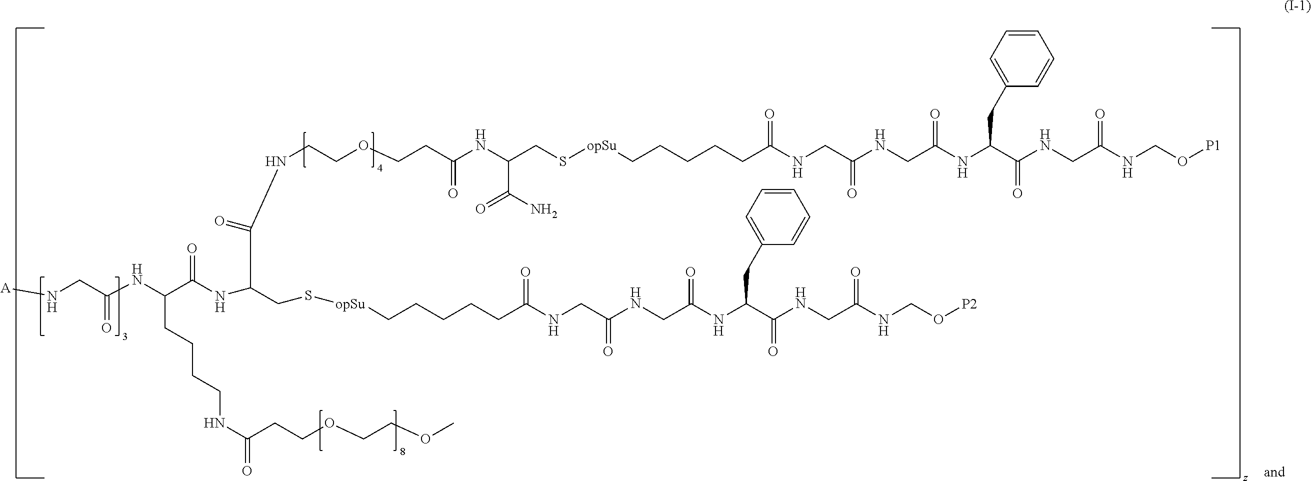

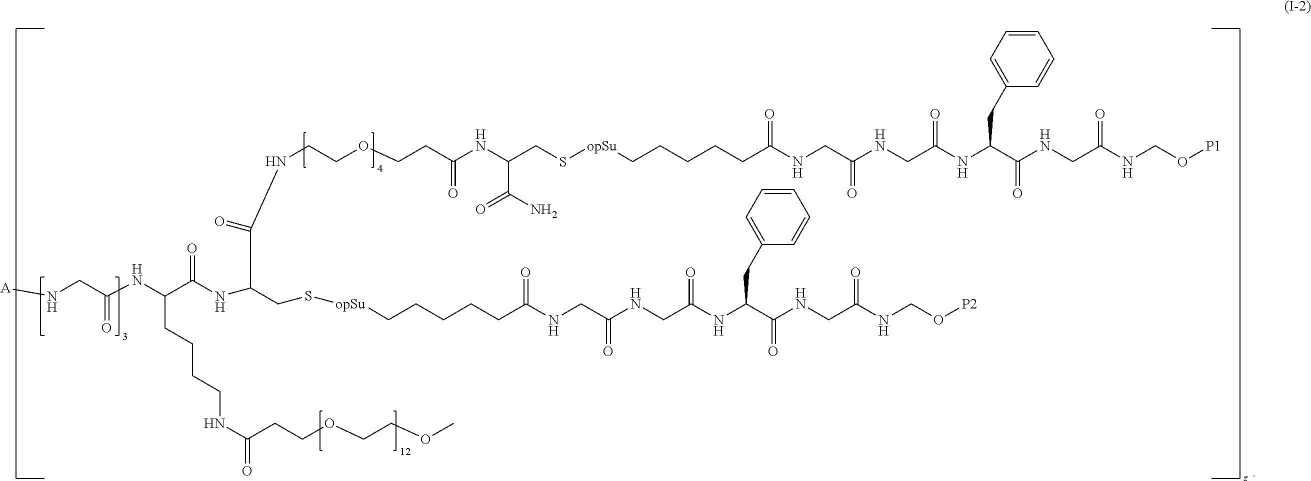

In an embodiment, the compound of formula (I) is selected from:

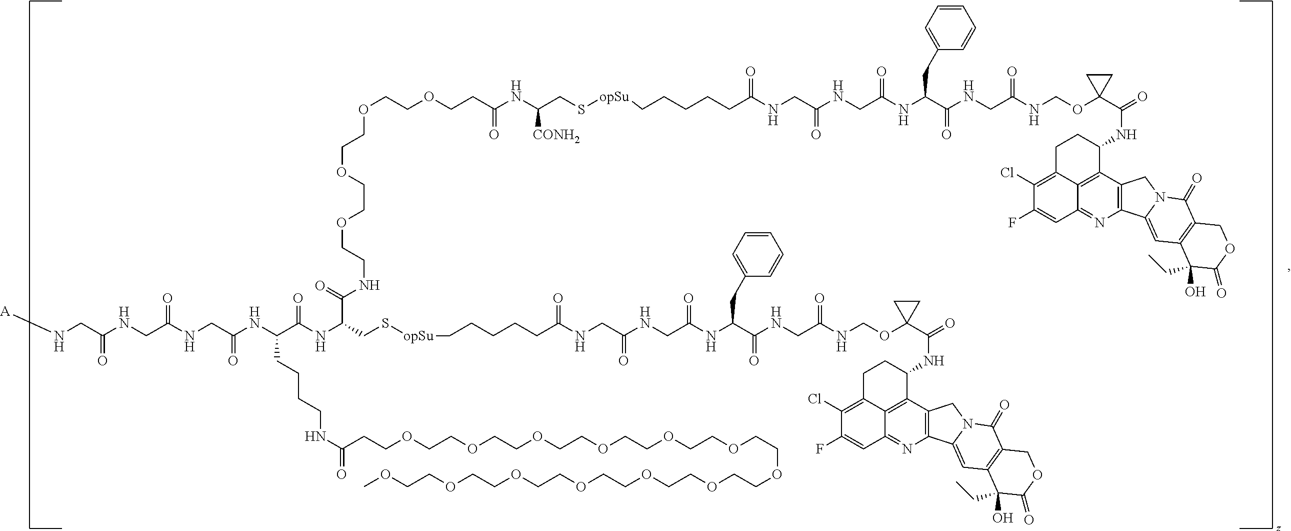

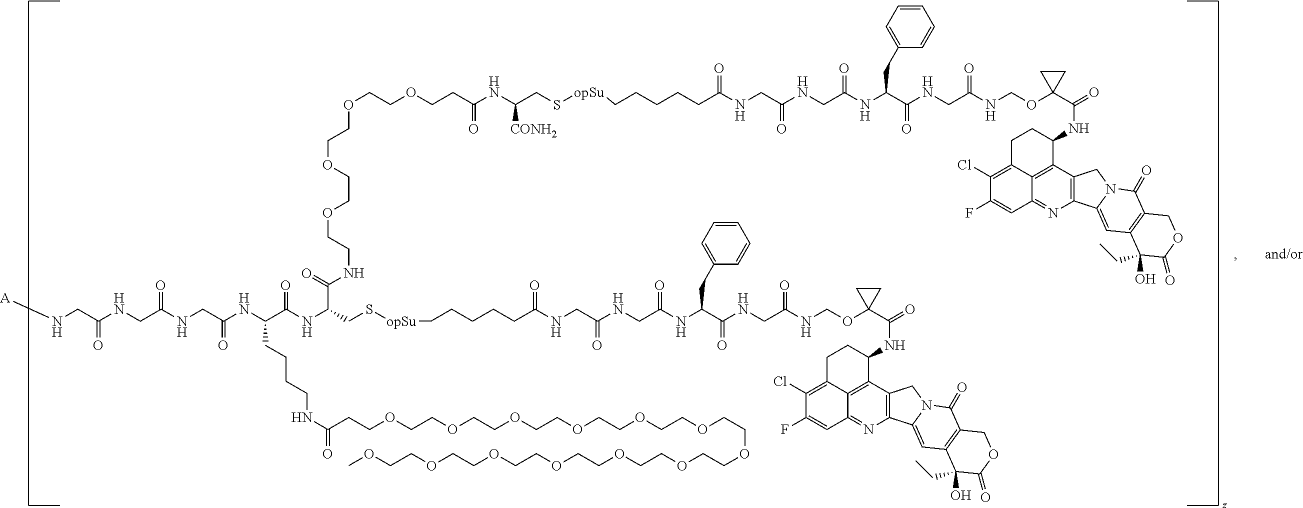

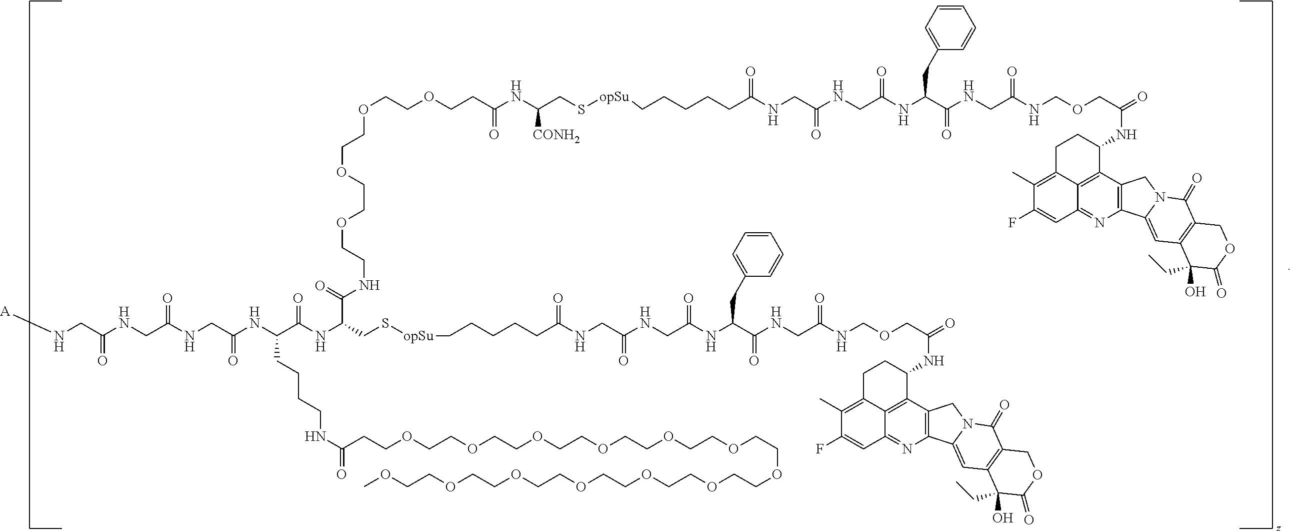

### Antibody or the Antigen Binding Fragment

In an embodiment, the antibody or the antigen binding fragment (shown as “A” in formula (I)) is an anti-FGFR3 antibody or an antigen binding fragment thereof. In some embodiments, the antibody or an antigen binding fragment comprises CDRs: a heavy chain CDR1 (HCDR1) comprising amino acid sequence of SEQ ID NO: 1 or having one to three conservative amino acid substitutions compared to SEQ ID NO: 1, a heavy chain CDR2 (HCDR2) comprising amino acid sequence of SEQ ID NO: 2 or having one to three conservative amino acid substitutions compared to SEQ ID NO: 2, a heavy chain CDR3 (HCDR3) comprising amino acid sequence of SEQ ID NO: 3 or having one to three conservative amino acid substitutions compared to SEQ ID NO: 3, a light chain CDR1 (LCDR1) comprising amino acid sequence of SEQ ID NO: 4 or having one to three conservative amino acid substitutions compared to SEQ ID NO: 4, a light chain CDR2 (LCDR2) comprising amino acid sequence of SEQ ID NO: 5 or having one to three conservative amino acid substitutions compared to SEQ ID NO: 5, and a light chain CDR3 (LCDR3) comprising amino acid sequence of SEQ ID NO: 6 or having one to three conservative amino acid substitutions compared to SEQ ID NO: 6.

In some embodiments, the antibody or the antigen binding fragment comprises HCDR1 of SEQ ID NO: 1, HCDR2 of SEQ ID NO: 2, HCDR3 of SEQ ID NO: 3, LCDR1 of SEQ ID NO: 4, LCDR2 of SEQ ID NO: 5, LCDR3 of SEQ ID NO: 6.

In some embodiments, the KD value of the anti-FGFR3 antibody or an antigen binding fragment binding to human FGFR3 and/or monkey FGFR3 is less than 10 nM. In some embodiments, the KD value of the anti-FGFR3 antibody or an antigen binding fragment binding to human FGFR3 and/or monkey FGFR3 is about 9.9 nM, about 9 nM, about 8 nM, about 7 nM, about 6 nM, about 5 nM, about 4.4 nM, about 3.9 nM, about 2 nM, about 1 nM, about 0.9 nM, about 0.7 nM, about 0.5 nM, about 0.3 nM, about 0.2 nM, about 0.1 nM, or the range between any two values (including the end value).

In some embodiments, the antibody or an antigen binding fragment comprises a heavy chain variable domain (VH) comprising an amino acid sequence having at least about 90% sequence identity to the amino acid sequence of SEQ ID NO: 7, and/or a light chain variable domain (VL) comprising an amino acid sequence having at least about 90% sequence identity to the amino acid sequence of SEQ ID NO: 8. In some embodiments, the antibody or an antigen binding fragment comprises a heavy chain variable domain comprising an amino acid sequence having at least about 91%, about 92%, about 93%, about 94%, about 95%, about 96%, about 97%, about 98%, about 99%, about 100% (the range between any two values (including the end value)) sequence identity to the amino acid sequence of SEQ ID NO: 7, and the antibody or an antigen binding fragment comprises a light chain variable domain comprising an amino acid sequence having at least about 91%, about 92%, about 93%, about 94%, about 95%, about 96%, about 97%, about 98%, about 99%, about 100% (the range between any two values (including the end value)) sequence identity to the amino acid sequence of SEQ ID NO: 8. In some embodiments, the antibody or an antigen binding fragment comprises VH of SEQ ID NO: 7, and/or VL of SEQ ID NO: 8. In some embodiments, the antibody or an antigen binding fragment comprises VH of SEQ ID NO: 7, and VL of SEQ ID NO: 8.

In some embodiments, the antibody or an antigen binding fragment comprises a heavy chain constant domain (CH) comprising an amino acid sequence having at least about 90% sequence identity to the amino acid sequence of SEQ ID NO: 9 or SEQ ID NO: 10, and/or a light chain constant domain (CL) comprising an amino acid sequence having at least 90% sequence identity to the amino acid sequence of SEQ ID NO: 11. In some embodiments, the antibody or an antigen binding fragment comprises a heavy chain constant domain comprising an amino acid sequence having at least about 90%, about 91%, about 92%, about 93%, about 94%, about 95%, about 96%, about 97%, about 98%, about 99%, about 100% (the range between any two values (including the end value)) sequence identity to the amino acid sequence of SEQ ID NO: 9 or SEQ ID NO: 10, and a light chain constant domain comprising an amino acid sequence having at least about 90%, about 91%, about 92%, about 93%, about 94%, about 95%, about 96%, about 97%, about 98%, about 99%, about 100% (the range between any two values (including the end value)) sequence identity to the amino acid sequence of SEQ ID NO: 11. In some embodiments, the antibody or an antigen binding fragment comprises a heavy chain constant domain of SEQ ID NO: 9, and a light chain constant domain of SEQ ID NO: 11. In some embodiments, the antibody or an antigen binding fragment comprises a heavy chain constant domain of SEQ ID NO: 10, and a light chain constant domain of SEQ ID NO: 11.

In some embodiments, the antibody or an antigen binding fragment comprises a heavy chain comprising an amino acid sequence having at least about 90% sequence identity to the amino acid sequence of SEQ ID NO: 12 or SEQ ID NO: 13, and/or a light chain comprising an amino acid sequence having at least about 90% sequence identity to the amino acid sequence of SEQ ID NO: 14. In some embodiments, the antibody or an antigen binding fragment comprises a heavy chain comprising an amino acid sequence having at least about 90%, about 91%, about 92%, about 93%, about 94%, about 95%, about 96%, about 97%, about 98%, about 99%, about 100% (the range between any two values (including the end value)) sequence identity to the amino acid sequence of SEQ ID NO: 12 or SEQ ID NO: 13, and/or a light chain comprising an amino acid sequence having at least about 90%, about 91%, about 92%, about 93%, about 94%, about 95%, about 96%, about 97%, about 98%, about 99%, about 100% (the range between any two values (including the end value)) sequence identity to the amino acid sequence of SEQ ID NO: 14. In some embodiments, the antibody or an antigen binding fragment comprises a heavy chain of SEQ ID NO: 12 and a light chain comprising an amino acid sequence of SEQ ID NO: 14. In some embodiments, the antibody or an antigen binding fragment comprises a heavy chain of SEQ ID NO: 13 and a light chain comprising an amino acid sequence of SEQ ID NO: 14.

The above-mentioned sequences are listed below
(Divided according to Kabat):
SEQ ID NO: 1:
SYDMS
SEQ ID NO: 2:
GIYSGDGSIYYADSVKG
SEQ ID NO: 3:
DGPMNEESRFDY
SEQ ID NO: 4:
SGSSSNIGNNYVS
SEQ ID NO: 5:
ADSKRPS
SEQ ID NO: 6:
ASWDYSLSGYV
SEQ ID NO: 7:
EVQLLESGGGLVQPGGSLRLSCAASGFTFSSYDMSWVRQAPGKGLEWVSGIYSGDGS
IYYADSVKGRFTISRDNSKNTLYLQMNSLRAEDTAVYYCAKDGPMNEESRFDYWGQ
GTLVTVSS
SEQ ID NO: 8:
QSVLTQPPSASGTPGQRVTISCSGSSSNIGNNYVSWYQQLPGTAPKLLIYADSKRPSGV
PDRFSGSKSGTSASLAISGLRSEDEADYYCASWDYSLSGYVFGGGTKLTVLG
SEQ ID NO: 9:
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 10:
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSP
SEQ ID NO: 11:
QPKAAPSVTLFPPSSEELQANKATLVCLISDFYPGAVTVAWKADSSPVKAGVETTTPSK
QSNNKYAASSYLSLTPEQWKSHRSYSCQVTHEGSTVEKTVAPTEC
SEQ ID NO: 12:
EVQLLESGGGLVQPGGSLRLSCAASGFTFSSYDMSWVRQAPGKGLEWVSGIYSGDGS
IYYADSVKGRFTISRDNSKNTLYLQMNSLRAEDTAVYYCAKDGPMNEESRFDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 13:
EVQLLESGGGLVQPGGSLRLSCAASGFTFSSYDMSWVRQAPGKGLEWVSGIYSGDGS
IYYADSVKGRFTISRDNSKNTLYLQMNSLRAEDTAVYYCAKDGPMNEESRFDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSP
SEQ ID NO: 14:
QSVLTQPPSASGTPGQRVTISCSGSSSNIGNNYVSWYQQLPGTAPKLLIYADSKRPSGV
PDRFSGSKSGTSASLAISGLRSEDEADYYCASWDYSLSGYVFGGGTKLTVLGQPKAA
PSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTEC

In an embodiment, the antibody or the antigen-binding fragment thereof may comprise terminal modification. A terminal modification refers to a modification at the C-terminal or N-terminal of the heavy chain or light chain of the antibody, which for example comprises a ligase recognition sequence. In another embodiment, the terminal modification may further comprise a spacer Sp comprising 2-100 amino acids, wherein the antibody, Sp and the ligase recognition sequence are sequentially linked. In a preferred embodiment, Sp is a spacer sequence containing 2-20 amino acids. In a particular embodiment, Sp is a spacer sequence selected from GA, GGGGS (SEQ ID NO: 17), GGGGSGGGGS (SEQ ID NO: 18) and GGGGSGGGGSGGGGS (SEQ ID NO: 19), especially GA.

In some embodiments, the modified antibody or the antigen-binding fragment thereof comprises a heavy chain of SEQ ID NO: 15, and/or a light chain of SEQ ID NO: 16. In some embodiments, the modified antibody or the antigen-binding fragment thereof comprises a heavy chain of SEQ ID NO: 15 and a light chain of SEQ ID NO: 16. In some embodiments, the modified antibody or the antigen-binding fragment thereof comprises a heavy chain of SEQ ID NO: 15 and a light chain of SEQ ID NO: 14. In some embodiments, the modified antibody or the antigen-binding fragment thereof comprises a heavy chain of SEQ ID NO: 12 and a light chain of SEQ ID NO: 16.

SEQ ID NO: 15:
EVQLLESGGGLVQPGGSLRLSCAASGFTFSSYDMSWVRQAPGKGLEWVSGIYSGDGS
IYYADSVKGRFTISRDNSKNTLYLQMNSLRAEDTAVYYCAKDGPMNEESRFDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGALPETGG
SEQ ID NO: 16:
QSVLTQPPSASGTPGQRVTISCSGSSSNIGNNYVSWYQQLPGTAPKLLIYADSKRPSGV
PDRFSGSKSGTSASLAISGLRSEDEADYYCASWDYSLSGYVFGGGTKLTVLGQPKAA
PSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECGALPETGG

### Preparation of the Conjugate

The conjugates (i.e., the compound of formula (I)) of the present disclosure can be prepared by any method known in the art. In some embodiments, the conjugate is prepared by the ligase-catalyzed site-specific conjugation of an antibody or an antigen binding fragment and a compound of formula (III), wherein the antibody or the antigen binding fragment thereof is modified by a ligase recognition sequence.

The antibody or the antigen binding fragment thereof and the compound of formula (III) are linked to each other via the ligase-specific recognition sequences of the substrates. The recognition sequence depends on the particular ligase employed. In an embodiment, the antibody or the antigen binding fragment thereof is an antibody with recognition sequence-based terminal modifications introduced at the C-terminal of the light chain and/or C-terminal of the heavy chain, and the antibody or the antigen binding fragment thereof is conjugated with the compound of formula (I), under the catalysis of the wild type or optimized engineered ligase or any combination thereof, and under suitable catalytic reaction conditions.

In a specific embodiment, the ligase is Sortase A and the conjugation reaction can be represented by the following scheme:

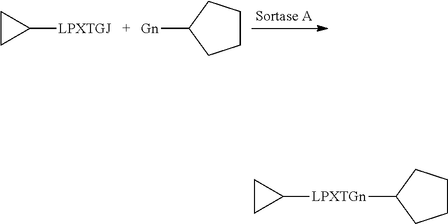

The triangle represents a portion of an antibody; the pentagon represents a portion of a compound of formula (III); and Gₙ represents the (Gly)ₙ moiety. n, X and J are respectively as defined above. When conjugated with Gₙ, which is the corresponding recognition sequence of the acceptor substrate, the upstream peptide bond of the glycine in the LPXTGJ sequence is cleaved by Sortase A, and the resulting intermediate is linked to the free N-terminal of Gₙ to generate a new peptide bond. The resulting amino acid sequence is LPXTGₙ. The sequences Gₙ and LPXTGJ are as defined above.

The compound of formula (III) of the present disclosure has defined structure, defined composition and high purity, so that when the conjugation reaction with an antibody is conducted, fewer impurities are introduced or no other impurities are introduced. When such an intermediate is used for the ligase-catalyzed site-specific conjugation with a modified antibody containing a ligase recognition sequence, a homogeneous ADC with highly controllable quality is obtained.

### Metabolism of the Conjugate in a Physiological Environment

When a part or whole linker is cleaved in tumor cells, the payload is released. As the linker is cleaved at a connecting position to the antitumor compound, the antitumor compound is released in its intrinsic structure to exhibit its intrinsic antitumor effect.

In an embodiment, the GGFG (Gly-Gly-Phe-Gly) (SEQ ID NO: 27) moiety comprised by the compound of formula III) can be cleaved by lysosomal enzymes (such as cathepsin B and/or cathepsin L).

In an embodiment, the compound of formula (III) comprises a self-immolative spacer. In an embodiment, the self-immolative spacer is an acetal or a heteroacetal. In an embodiment, the -GGFG-NH—CH₂—O— moiety comprised by the compound of formula (III) represents a combination of a restriction enzyme site and a self-immolative spacer, which would cleave in the cell and release the aimed molecule (such as the antitumor compound).

### Pharmaceutical Composition and Pharmaceutical Preparation

Another object of the disclosure is to provide a pharmaceutical composition comprising the conjugate of the present disclosure, and at least one pharmaceutically acceptable carrier.

The pharmaceutical composition of the present disclosure may be administered in any manner as long as it achieves the effect of preventing, alleviating, preventing or curing the symptoms of a human or animal. For example, various suitable dosage forms can be prepared according to the administration route, especially injections such as lyophilized powder for injection, injection, or sterile powder for injection.

The term “pharmaceutically acceptable” means that when contacted with tissues of the patient within the scope of normal medical judgment, no undue toxicity, irritation or allergic reaction, etc. shall arise, having reasonable advantage-disadvantage ratios and effective for the intended use.

The term pharmaceutically acceptable carrier refers to those carrier materials which are pharmaceutically acceptable and which do not interfere with the bioactivities and properties of the conjugate. Examples of aqueous carriers include but are not limited to buffered saline, and the like. The pharmaceutically acceptable carrier also includes carrier materials which brings the composition close to physiological conditions, such as pH adjusting agents, buffering agents, toxicity adjusting agents and the like, and sodium acetate, sodium chloride, potassium chloride, calcium chloride, sodium lactate, and the like. In some embodiments, The term “carrier” refers to a diluent, adjuvant, excipient, or vehicle that is administered with an active ingredient for treatment. Such pharmaceutical carriers may be sterile liquids, such as water and oils, including oils originated from petroleum, animal, plant or synthesis, such as peanut oil, soybean oil, mineral oil and sesame oil. Water is a preferred carrier when the pharmaceutical composition is administered intravenously. Saline and solutions of glucose in water or glycerol can also be used as a liquid carrier, particularly for injection. Suitable pharmaceutical excipients include starch, glucose, lactose, sucrose, gelatin, malt, rice, flour, chalk, silica gel, sodium stearate, glycerol monostearate, talc, sodium chloride, skimmed milk powder, glycerol, propylene, glycol, water, ethanol and the like. If desired, the composition may also comprise a small amount of a wetting agent, an emulsifier, or a pH buffering agent such as acetates, citrates or phosphates. Antibacterials such as benzyl alcohol or methylparaben, antioxidants such as ascorbic acid or sodium bisulfite, chelating agents such as ethylenediamine tetraacetic acid, and tonicity adjusting agents such as sodium chloride or dextrose are also contemplated. Such compositions may be in the form of solutions, suspensions, emulsions, tablets, pills, capsules, pulvises, sustained-release formulations and the like. The composition may be formulated as a suppository using conventional binders and carriers such as triglycerides. Oral formulations may comprise standard carriers such as mannitol, lactose, starch, magnesium stearate, sodium saccharin, cellulose and magnesium carbonate of pharmaceutical grade. Examples of suitable pharmaceutical carriers are described in Remington's Pharmaceutical Sciences by E. W. Martin, which is incorporated herein by reference. Such composition will comprise a clinically effective dose of an antibody, preferably in purified form, together with a suitable amount of a carrier to provide a dosing form suitable for the patient. The formulation should be suitable for the administration mode. The parent formulation may be enclosed in ampoules, disposable syringes or multiple dose vials made of glass or plastic.

In an embodiment, the pharmaceutical composition of the present disclosure has a drug to antibody ratio (DAR) of an integer or non-integer of about 1 to about 20, such as about 1 to about 10, about 1 to about 8, about 1 to about 6, about 1 to about 4. In a particular embodiment, the conjugate of the present disclosure has a DAR of about 4.

### Treatment Method and Use

The conjugates of the present disclosure are useful for the treatment of FGFR3-mediated disease.

Accordingly, in yet another aspect, also provided is use of a conjugate of the present disclosure or a pharmaceutical composition of the present disclosure in the manufacture of a medicament for treating FGFR3-mediated disease. Specifically an FGFR3-positive tumor, more specifically brain cancer, bladder cancer, urothelial cancer, cervical cancer, or intrahepatic cholangiocarcinoma.

In some embodiments, the disease includes tumor overexpressing FGFR3 or tumor with FGFR3 gene mutation. In some embodiments, the disease is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendothelial sarcoma, synovioma, mesothelioma, Ewing's tumor, leiomyosarcoma, rhabdomyosarcoma, colon cancer, pancreatic cancer, breast cancer, thyroid cancer, endometrial cancer, melanoma, ovarian cancer, prostate cancer, squamous cell carcinoma, basal cell carcinoma, adenocarcinoma, sweat gland carcinoma, sebaceous carcinoma, papillary carcinoma, papillary adenocarcinoma, cystadenocarcinoma, medullary carcinoma, bronchial carcinoma, renal cell carcinoma, liver cancer, bile duct cancer, choriocarcinoma, seminoma, embryonal carcinoma, Wilms' tumor, cervical cancer, testicular tumor, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, neuroblastoma and retinoblastoma. In some embodiments, the disease is selected from: brain cancer, bladder cancer, urothelial cancer, cervical cancer, or intrahepatic cholangiocarcinoma. In some embodiments, the disease is glioblastoma. In a preferred embodiment, the conjugate of the present disclosure formed by conjugation of the anti-FGFR3 antibody and the small molecule cytotoxin can specifically bind to FGFR3 on the surface of the tumor cell and selectively kill the FGFR3-expressing tumor cells.

The dosage of the conjugate administered to the subject can be adjusted to a considerable extent. The dosage can vary according to the particular route of administration and the needs of the subject, and can be subjected to the judgment of the health care professional.

### Beneficial Effects

The novel small molecule topoisomerase I inhibitors provided in the present disclosure, either used alone or as a component of ADCs, could show greater activity, stability, physiochemical properties over the prior art.

The antibody-drug conjugate of the present invention uses specially designed linker-payload, and can achieve great efficacy and bystander killing effects. At the same time, it has a lower DAR, and therefore can reduce side effects and increase the therapeutic index, which is of special importance for bystander killing. The antibody-drug conjugate of the present invention is more stable in its structure, such as the ring-opened succinimide structure.

The present disclosure utilizes a linker with unique structure and uses a ligase to catalyze the conjugation of the targeting molecule and the payload. The conjugate of the present disclosure has good homogeneity and high activity. Furthermore, the toxicity of the linker-payload intermediate is much lower than that of the free payload, and thus the manufacture process of the drug is less detrimental, which is advantageous for industrial production.

The conjugate of the present disclosure achieves at least one of the following technical effects:
(1) High inhibitory activity against target cells, or strong killing effect on target cells.
(2) Good physicochemical properties (e.g., solubility, physical and/or chemical stability, wherein the chemical stability includes the low fall-off rate of the cytotoxin from the ADC which leads to the low off-target toxicity).
(3) Good pharmacokinetic properties (e.g., good stability in plasma, appropriate half-life and duration of action).
(4) High specificity and good safety (low toxicity on non-target normal cells or tissues, and/or fewer side effects, wider treatment window), etc.
(5) Highly modular design, simple assembly of multiple drugs.

### Examples

In order to more clearly illustrate the objects and technical solutions, the present disclosure is further described below with reference to specific examples. It is to be understood that the examples are not intended to limit the scope of the disclosure. The specific experimental methods which were not mentioned in the following examples were carried out according to conventional experimental method.

Unless otherwise stated, the instruments and reagents used in the examples are commercially available. The reagents can be used directly without further purification.
MS: Thermo Fisher Q Exactive Plus, Waters 2795-Quattro micro triple quadrupole mass spectrometer
HPLC: Waters 2695, Agilent 1100, Agilent 1200
Semi-preparative HPLC: Lisure HP plus 50D
Flow Cytometry: CytoFLEX S
HIC-HPLC: Butyl-HIC; mobile phase A: 25 mM PB, 2M (NH₄)₂SO₄, pH 7.0; mobile phase B: 25 mM PB, pH 7.0; flow rate: 0.8 ml/min; acquisition time: 25 min; injection amount: 20 μg; column temperature: 25° C.; detection wavelength: 280 nm; sample chamber temperature: 8° C.
SEC-HPLC: column: TSK-gel G3000 SWXL, TOSOH 7.8 mm ID×300 mm, 5 μm;
mobile phase: 0.2 M KH₂PO₄, 0.25 M KCl, pH 6.2; flow rate: 0.5 ml/min; acquisition time: 30 min; injection volume: 50 μl; column temperature: 25° C.; detection wavelength; 280 nm; sample tray temperature: 8° C.

In some cases, the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Example 1 Preparation of Linker-Payload 1

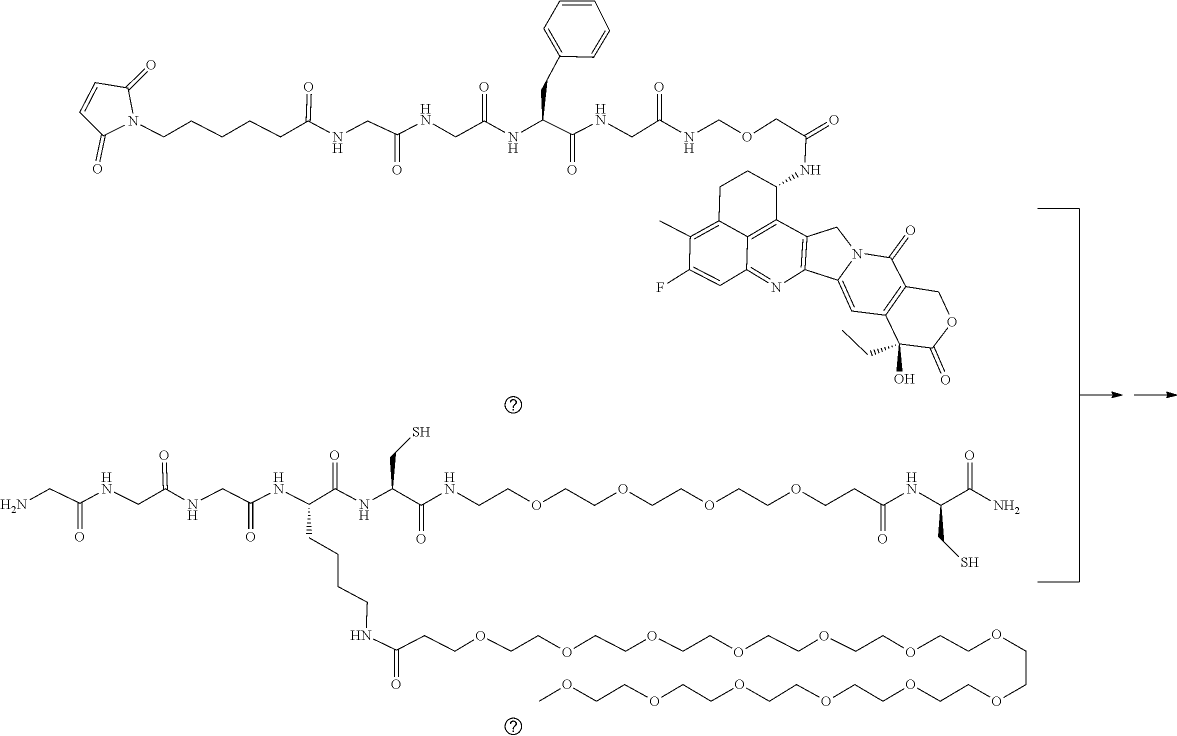 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; COCCOCCOCCOCCOCCOCCOCCOCCOCCOCCOCCOCCOCCC(=O)NCCCC[C@H](NC(=O)CNC(=O)CNC(=O)CN)C(=O)N[C@@H](CS)C(=O)NCCOCCOCCOCCOCCC(=O)N[C@H](CS)C(N)=O]

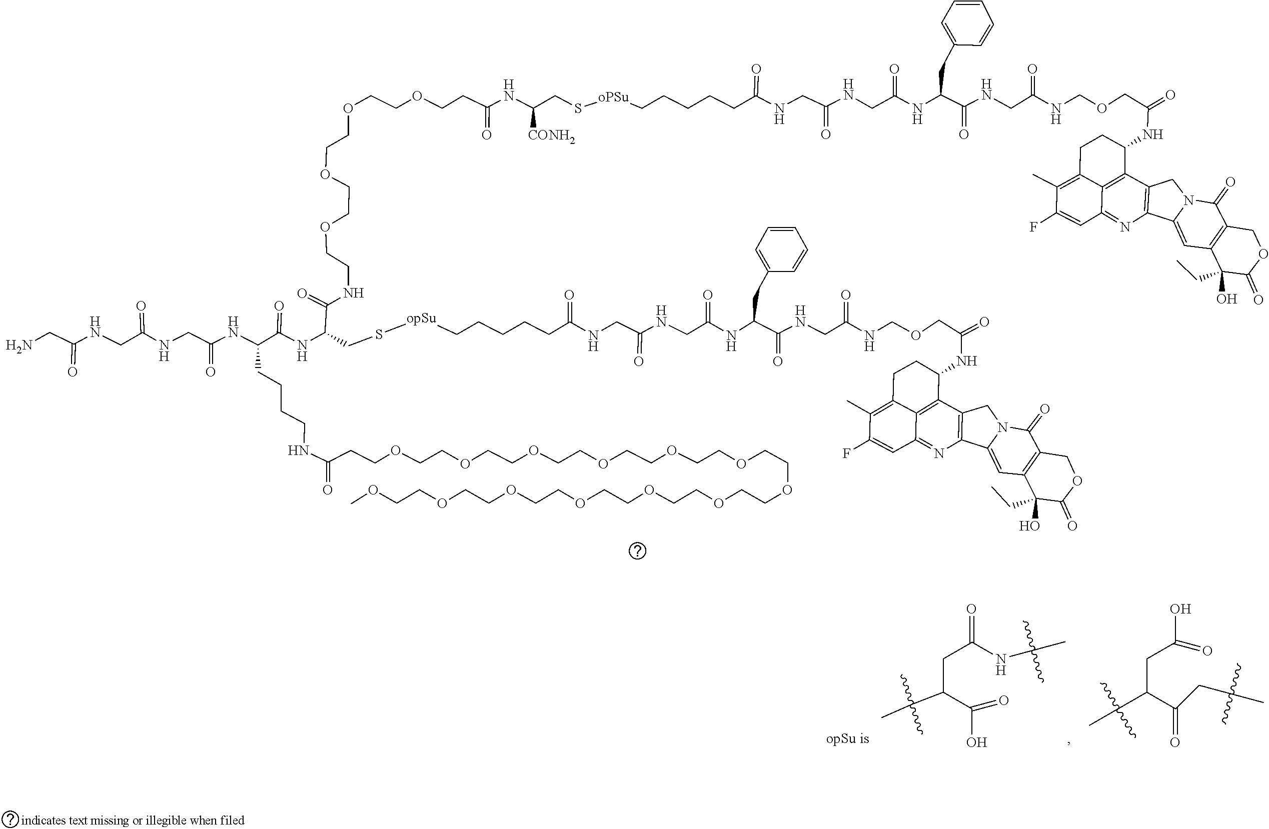

or a mixture thereof;

### Preparation of Intermediate MC-GGFG-DXd

The intermediate MC-GGFG-DXd is commercial available or prepared following the procedures as described in EP2907824. This compound is used to prepare linker-payload 1.

### Preparation of Linker-Payload Intermediate 1

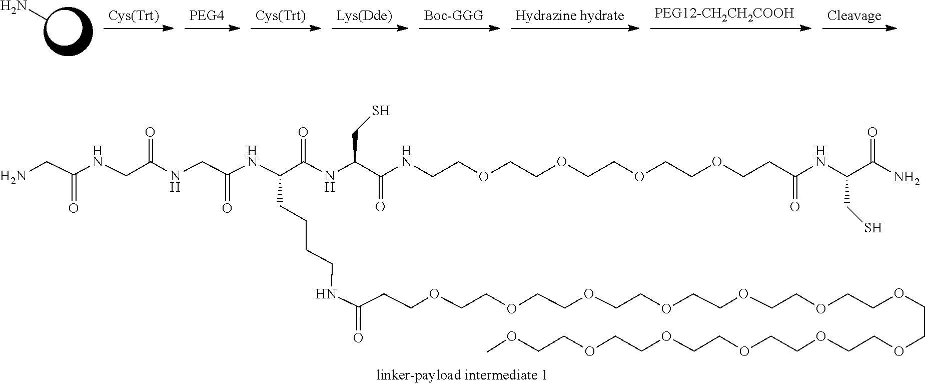

Linker-payload intermediate 1 can be synthesized by a conventional solid phase polypeptide synthesis using Rink-amide-MBHA-resin. Fmoc was used to protect the amino acid in the linking unit. The coupling reagent was selected from HOBT, HOAt/DIC, DCC, EDCI or HATU. After synthesis, the product was cleaved from resin using TFA/TIS/H₂O solution. The product was purified by prep-HPLC, lyophilized and stored for use. LCMS m/z. [M−H]⁻=1382.6.

### Preparation of Linker-Payload 1

Linker-payload intermediate 1 and MC-GGFG-DXd (molar ratio˜1:2) were weighed and dissolved in water and DMF, respectively, and then thoroughly mixed to give a mixture, which was reacted at 0-40° C. for 0.5-30h. Once the reaction was completed, the reaction mixture was directly added with an appropriate amount of Tris Base solution or other solution that promotes the ring-opening reaction, and the reaction was performed at 0-40° C. for another 0.2-20 h. After the reaction was completed, the product was purified by semi-preparative/preparative HPLC and lyophilized to obtain linker-payload 1. LCMS m/z: [(M+3H)/3]⁺=1163.3.

### Example 2 Preparation of Linker-Payload 2

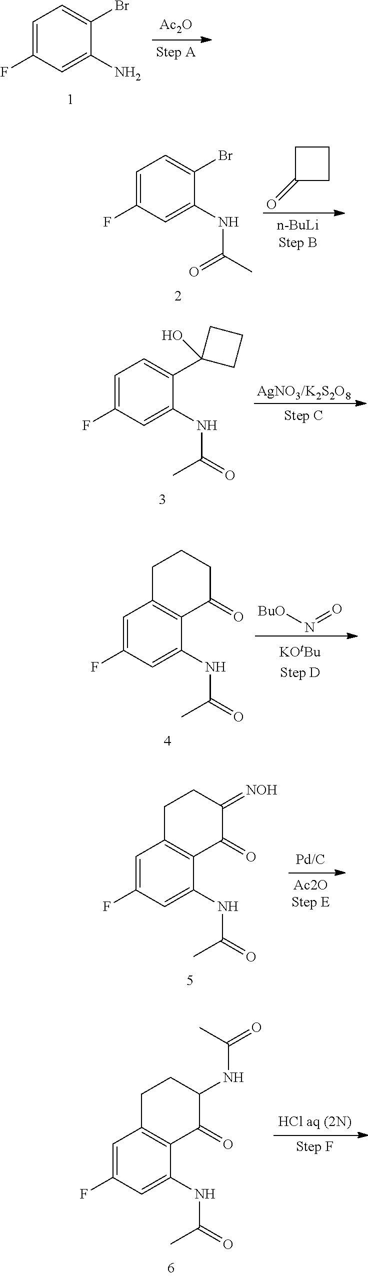

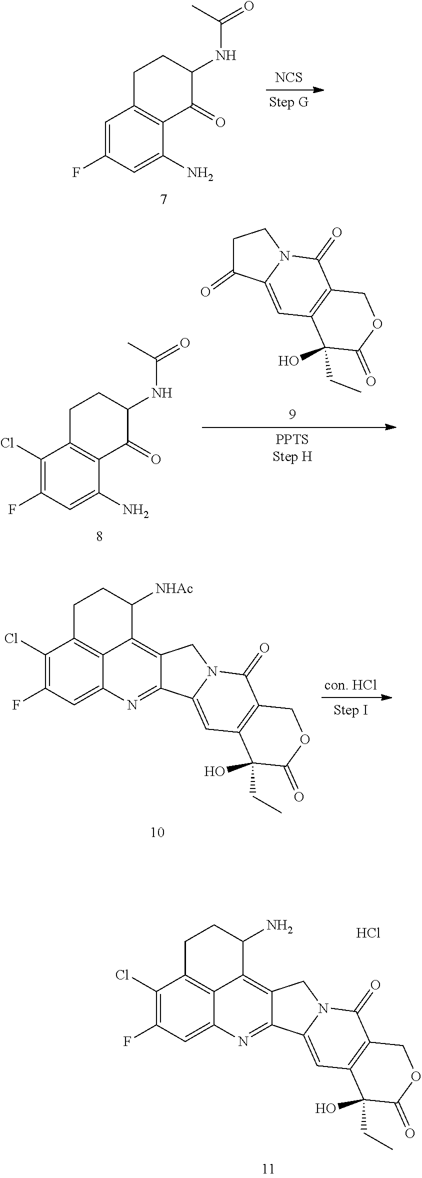

Step A: N-(2-bromo-5-fluorophenyl)acetamide: To a stirred solution of acetic anhydride (214 g, 2.10 mol) in acetic acid (500 mL) was added con. H₂SO₄ (3 mL), followed with 2-bromo-5-fluoroaniline (100 g, 526.27 mmol) in portions at room temperature. The mixture was stirred for 3 h, then poured into 2000 mL ice-water. A precipitate was formed, which was collected by filtration and dried in vacuo at room temperature to afford N-(2-bromo-5-fluorophenyl)acetamide (105 g) as a yellow solid. ¹H NMR (400 MHz, DMSO-d₆) δ 7.68 (dd, J=8.9, 6.0 Hz, 1H), 7.61 (ddd, J=10.7, 5.3, 3.1 Hz, 1H), 7.02 (ddd, J=8.9, 8.0, 3.1 Hz, 1H), 2.11 (s, 3H). LCMS m/z 232.0 (M+H).

Step B: N-(5-fluoro-2-(1-hydroxycyclobutyl)phenyl)acetamide: To a stirred solution of N-(2-bromo-5-fluorophenyl)acetamide (105 g, 452.48 mmol) in THF (1000 mL) was added n-BuLi (594 mL, 1.6 M in n-hexane, 950.22 mmol) dropwise over 1 h at −78° C. After completion, the mixture was stirred for 0.5 h under N₂. Then a solution of cyclobutanone (38.06 g, 542.98 mmol) in THF (50 mL) was added dropwise at −78° C. over 0.5 h, the mixture was stirred at −78° C. to room temperature for 6 h. The mixture was poured into 500 mL saturated NH₄Cl aq at 0° C. Extracted with ethyl acetate (500 mL×3), washed with brine (250 mL×2), dried over Na₂SO₄ and concentrated. The mixture was triturated with (PE/EA=1:1, 100 mL) for 10 mins, filtered and the cake was collected and dried in vacuo to afford N-(5-fluoro-2-(1-hydroxycyclobutyl)phenyl)acetamide (24 g) as a yellow solid. LCMS m/z 206.1 (M−18+H), 246.1 (M+Na).

Step C: N-(3-fluoro-8-oxo-5,6,7,8-tetrahydronaphthalen-1-yl)acetamide: To a stirred mixture of N-(5-fluoro-2-(1-hydroxycyclobutyl)phenyl)acetamide (24 g, 107.50 mmol) in CH₂Cl₂ (170 mL) and water (170 mL) was added silver nitrate (AgNO₃) (5.48 g, 32.25 mmol) and potassium persulfate (K₂S₂O₈) (58.12 g, 215.01 mmol), the mixture was stirred at 30° C. for 6 h. The mixture was filtered on Celite and washed with CH₂Cl₂ (100 mL), the filtrate was concentrated and purified by FCC(EA/PE=0-40%) to afford N-(3-fluoro-8-oxo-5,6,7,8-tetrahydronaphthalen-1-yl)acetamide (14 g) as a light yellow solid. LCMS m/z 222.1 (M+H).

Step D: N-(3-fluoro-7-(hydroxyimino)-8-oxo-5,6,7,8-tetrahydronaphthalen-1-yl)acetamide: To a stirring mixture of N-(3-fluoro-8-oxo-5,6,7,8-tetrahydronaphthalen-1-yl)acetamide (14 g, 63.28 mmol) in THF (500 mL) at 0° C. was added 1-butyl nitrite (8.48 g, 63.28 mmol), followed with t-BuOK (8.52 g, 75.94 mmol). The mixture was stirred at 0° C. for 2 h. After completion, the mixture was acidified by HCl (2 N) to adjust pH=3. The mixture was extracted by ethyl acetate (200 mL×3), washed by brine (100 mL×2), dried over Na₂SO₄ and concentrated under reduced pressure. The crude mixture was triturated with tert-butyl methyl ether (200 mL) for 10 mins, filtered and the cake was collected and dried in vacuo to afford N-(3-fluoro-7-(hydroxyimino)-8-oxo-5,6,7,8-tetrahydronaphthalen-1-yl)acetamide (12 g) as a yellow solid. LCMS m/z 251.1 (M+H).

Step E: N,N′-(3-fluoro-8-oxo-5,6,7,8-tetrahydronaphthalene-1,7-diyl) diacetamide: To a solution of N-(3-fluoro-7-(hydroxyimino)-8-oxo-5,6,7,8-tetrahydronaphthalen-1-yl)acetamide (12 g, 47.96 mmol) in acetic anhydride (90 mL) and THF (90 mL) was added 10% Pd/C (1 g), the mixture was stirred at 25° C. under H₂ atmosphere for 16 h. After cooling to 0° C., Et₃N (20 mL) was added dropwise, the mixture was stirred at 0° C. for 1 h. Filtered on Celite, the filtrate was poured into ice-water (500 mL). Extracted with ethyl acetate (500 mL×3), washed with brine (250 mL×2), dried over Na₂SO₄ and concentrated. The residue was triturated with tert-butyl methyl ether (120 mL) for 10 mins, filtered and the cake was collected and dried in vacuo to give N,N′-(3-fluoro-8-oxo-5,6,7,8-tetrahydronaphthalene-1,7-diyl)diacetamide (7.9 g) as a yellow solid. LCMS m/z 279.1 (M+H).

Step F: N,N′-(3-fluoro-8-oxo-5,6,7,8-tetrahydronaphthalene-1,7-diyl)diacetamide: To a solution of N,N′-(3-fluoro-8-oxo-5,6,7,8-tetrahydronaphthalene-1,7-diyl)diacetamide (7.9 g, 28.39 mmol) in MeOH (150 mL) was added HCl aq (2 N, 150 mL), the mixture was stirred at 50° C. for 7 h. After cooling to 0° C., Sat. NaHCO₃ aq was added dropwise to adjust pH=8. Extracted with ethyl acetate (200 mL×3), washed with brine (200 mL×2), dried over Na₂SO₄ and concentrated under reduced pressure to give N,N′-(3-fluoro-8-oxo-5,6,7,8-tetrahydronaphthalene-1,7-diyl)diacetamide (6.0 g) as a yellow solid. ¹H NMR (400 MHz, Chloroform-d) δ 6.57 (s, 3H), 6.18 (td, J=11.1, 2.4 Hz, 2H), 4.52 (dt, J=13.3, 5.0 Hz, 1H), 3.13 (ddd, J=17.5, 13.0, 4.6 Hz, 1H), 3.00-2.81 (m, 1H), 2.69 (dtd, J=9.4, 4.6, 2.5 Hz, 1H), 2.09 (s, 3H), 1.79 (qd, J=13.0, 4.3 Hz, 1H). LCMS m/z 237.1 (M+H).

Step G: N-(8-amino-5-chloro-6-fluoro-1-oxo-1,2,3,4-tetrahydronaphthalen-2-yl)acetamide: To a solution of N,N′-(3-fluoro-8-oxo-5,6,7,8-tetrahydronaphthalene-1,7-diyl)diacetamide (4.0 g, 16.93 mmol) in DMF (80 mL) was added NCS (2.26 g, 16.93 mmol) in portions at 0° C., the mixture was stirred at room temperature for 16 h. The mixture was poured into 200 mL ice-water. A precipitate was formed, which was collected by filtration and dried in vacuo at room temperature to afford N-(8-amino-5-chloro-6-fluoro-1-oxo-1,2,3,4-tetrahydronaphthalen-2-yl)acetamide (4.0 g) as a yellow solid. ¹H NMR (400 MHz, DMSO-d₆) δ 8.11 (d, J=8.0 Hz, 1H), 7.71 (s, 2H), 6.62 (d, J=11.9 Hz, 1H), 4.53 (ddd, J=13.0, 8.0, 4.7 Hz, 1H), 3.18-3.04 (m, 1H), 2.91 (ddd, J=17.5, 12.4, 4.8 Hz, 1H), 2.21-2.08 (m, 1H), 1.99-1.83 (m, 4H). LCMS m/z 271.0 (M+H).

Step H: N-((9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)acetamide: To a mixture of N-(8-amino-5-chloro-6-fluoro-1-oxo-1,2,3,4-tetrahydronaphthalen-2-yl)acetamide (4.0 g, 14.78 mmol) in toluene (400 mL) was added (S)-4-ethyl-4-hydroxy-7,8-dihydro-TH-pyrano[3,4-f]indolizine-3,6,10(4H)-trione (4.28 g, 16.25 mmol), pyridinium p-Toluenesulfonate (1.11 g, 4.43 mmol) and o-cresol (10 mL), the mixture was heated to reflux under N₂ for 24 h. The solvent was removed by reduced pressure and the mixture was purified by FCC(THF/CH₂Cl₂=0-60%) to afford N-((9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)acetamide (4.1 g) as a brown solid. LCMS m/z 498.1 (M+H).

Step I: (9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione: A mixture of N-((9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)acetamide (2.0 g, 4.02 mmol) in 20 mL con. HCl aq was stirred at 70° C. under N₂ for 36 h. The mixture was concentrated under reduced pressure to give crude (9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione hydrochloride (2 g) as a brown solid. LCMS (ESI) m/z 456.1 (M+H).

### Preparation of Intermediate 12 (12-1, 12-2)

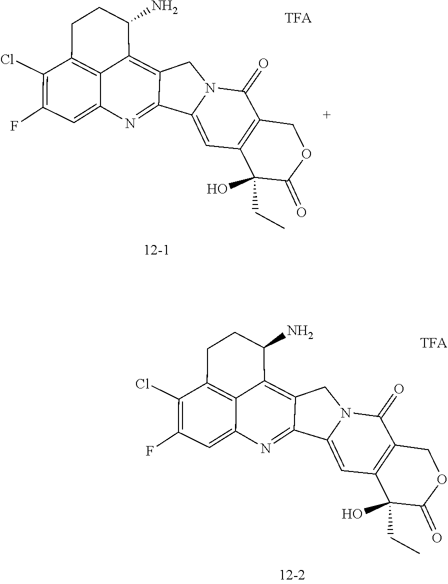 [SMILES: CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(Cl)c3c1c2[C@H](N)CC3]

12-1 and 12-2 were prepared by prep-HPLC from (9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione hydrochloride (intermediate 11) as TFA salt.

TABLE 1

| | | | | Retention |
|---|---|---|---|---|
| | | | | time on |
| | | | MS | HPLC |
| Number | Structure | ¹HNMR | (M + H) | (min) |
| 12-1 | | ¹H NMR (400 MHz, DMSO-d6) δ8.51 | 456.0 | 1.395 |
| | | (d, J = 4.8 Hz, 3H), 8.17 (d, J = 10.2 | | |
| | | Hz, 1H), 7.38 (s, 1H), 6.56 (s, 1H), | | |
| | | 5.74 (d, J = 19.4 Hz, 1H), 5.52 − 5.40 | | |
| | | (m, 3H), 5.16 (s, 1H), 3.44 (dd, J = | | |
| | | 16.3, 4.1 Hz, 1H), 3.19 (t, J = 13.9 Hz, | | |
| | | 1H), 2.57 (d, J = 14.0 Hz, 1H), 2.26 (t, | | |
| | | J = 14.3 Hz, 1H), 1.89 (hept, J = 7.0 | | |
| | | Hz, 2H), 0.89 (t, J = 7.3 Hz, 3H). | | |
| 12-2 | | ¹H NMR (400 MHz, DMSO-d6) δ | 456.0 | 1.541 |
| | | 8.56 (s, 2H), 8.17 (d, J = 10.1 Hz, 1H), | | |
| | | 7.38 (s, 1H), 6.57 (s, 1H), 5.74 (d, J = | | |
| | | 19.4 Hz, 1H), 5.53 − 5.28 (m, 3H), | | |
| | | 5.16 (s, 1H), 3.59 − 3.01 (m, 2H), 2.35 − | | |
| | | 2.19 (m, 2H), 2.00 − 1.78 (m, 2H), | | |
| | | 0.88 (t, J = 7.3 Hz, 3H). | | |

Conditions of HPLC above: Equipment: Agilent 1200; Chromatographic column: Waters XBridge C18 4.6*50 mm, 3.5 um; Flow: 2.0 mL/min; Gradient elute: 5.0%-95.0%-95.0%-5.0%-5.0%, 0.00 min-1.50 min-2.50 min-2.52 min-3.00 min; Temperature: 40° C.; Phase: A: Acetonitrile, B: H₂O (0.05% TFA); Wavelength: 214 nm/254 nm.

### Preparation of Linker-Payload 2

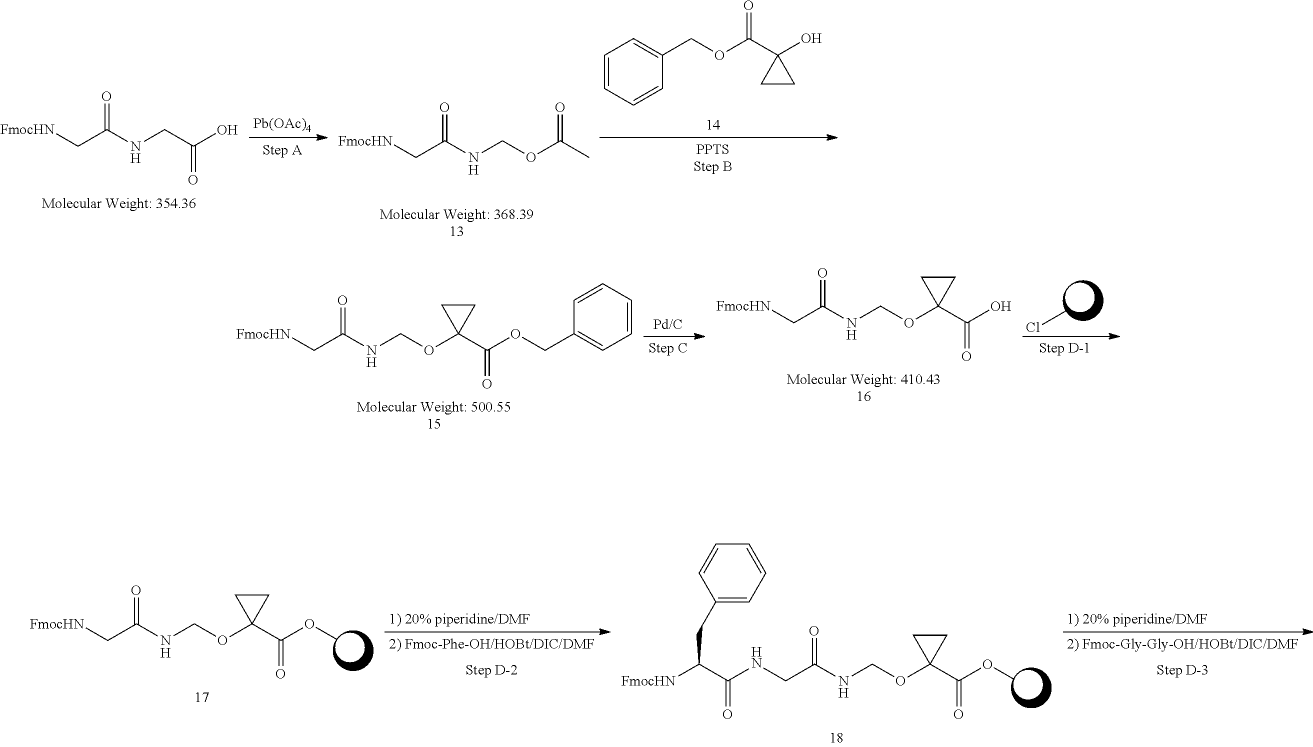

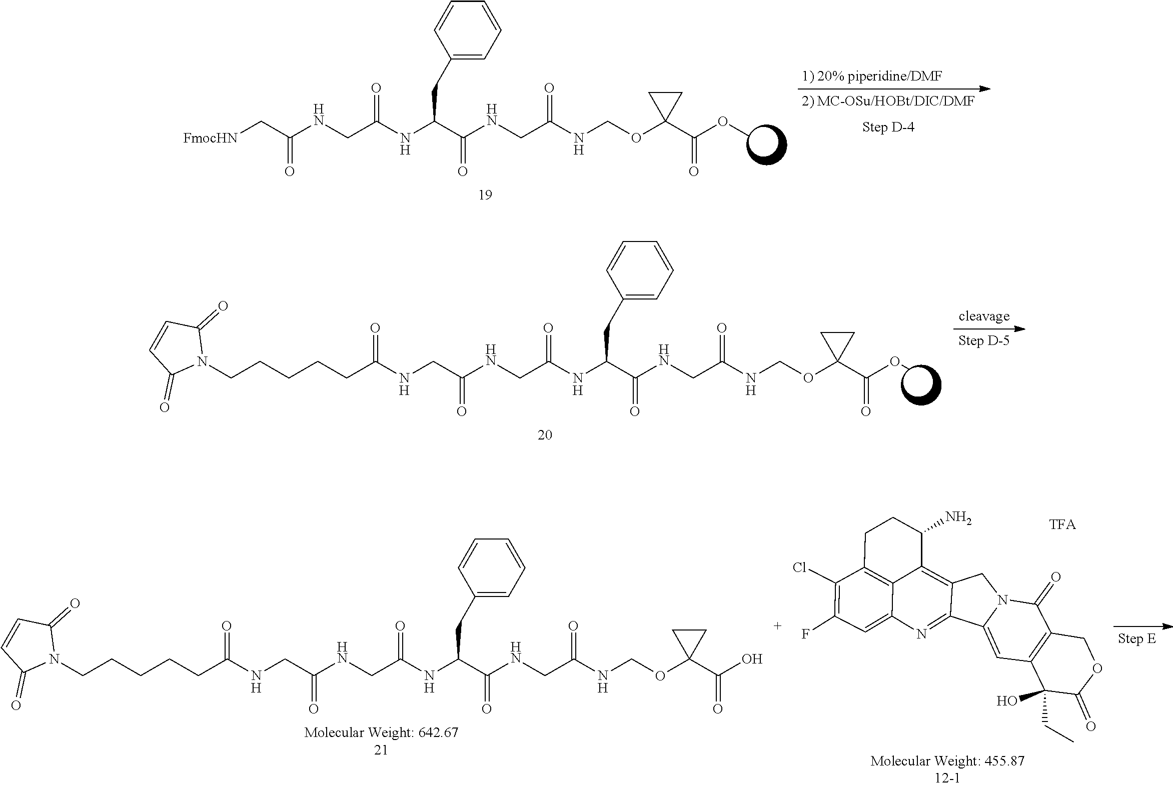

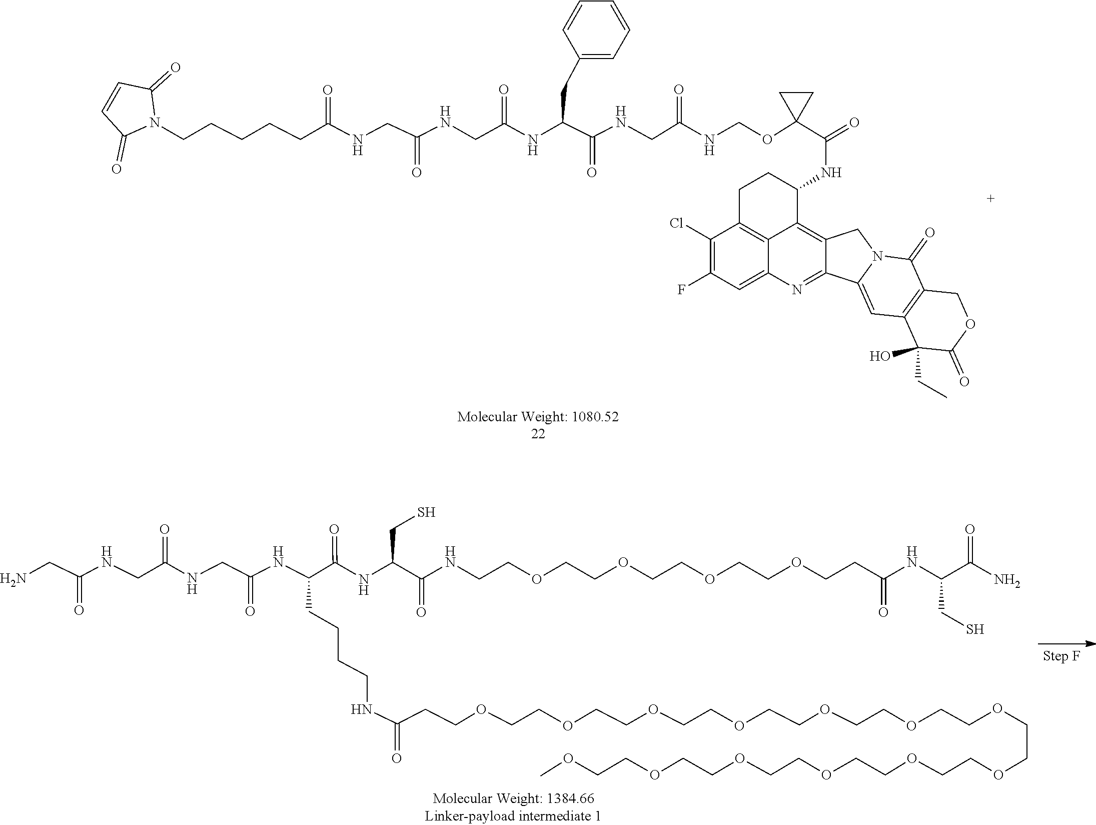 [SMILES: CC[C@@]1(O)C(=O)OCc2c1cc1n(c2=O)Cc2c-1nc1cc(F)c(Cl)c3c1c2[C@@H](NC(=O)C1(OCNC(=O)CNC(=O)[C@H](Cc2ccccc2)NC(=O)CNC(=O)CNC(=O)CCCCCN2C(=O)C=CC2=O)CC1)CC3 ; COCCOCCOCCOCCOCCOCCOCCOCCOCCOCCOCCOCCOCCC(=O)NCCCC[C@H](NC(=O)CNC(=O)CNC(=O)CN)C(=O)N[C@@H](CS)C(=O)NCCOCCOCCOCCOCCC(=O)N[C@@H](CS)C(N)=O]

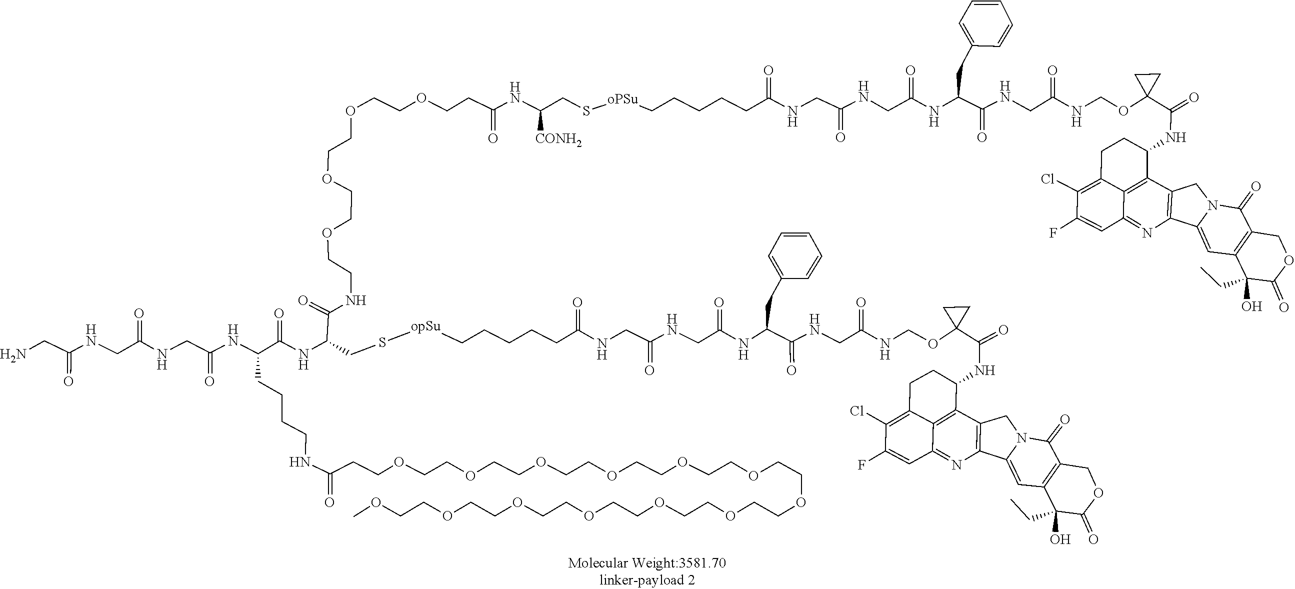

### Synthesis of 13 (Step A)

4.33 g Fmoc-Gly-Gly-OH and 6.84 g Pb(OAc)₄ were weighed and added into a 500 ml single-neck round bottom flask. Anhydrous THF/Toluene (120/40 ml) was added under nitrogen atmosphere and stirred for dissolving. Then 1.16 mL of pyridine was added to the reaction system. The reaction system was heated to 80° C. and refluxed for 5 hr under nitrogen atmosphere. Samples were taken and detected by HPLC to monitor the reaction.

The reaction system was cooled to room temperature, filtered, and the filter cake was washed with EA for 3 times. The filtrates were combined and concentrated to dryness. Column chromatography was performed (PE:EA=100:0˜50:100) to give about 2000 mg of the target product in white solid with a yield of 44%.

### Synthesis of 15 (Step B)

200 mg 13 was weighed and added into a 100 ml single-neck round bottom flask. Then 15 ml THF was added and stirred for dissolving. Then 14 (312 mg, 3.0 eq) and TsOH H₂O (15 mg, 0.15 eq) were added to the reaction system. The reaction system was reacted overnight at room temperature. Samples were taken and detected by TLC (PE/EA=1:1) to monitor the reaction. The raw material basically disappeared, and a new point was detected.

Saturated sodium bicarbonate solution was added to quench reaction. Extraction was conducted with EA for 3 times. The organic phase was combined and washed with saline, dried with anhydrous magnesium sulfate and concentrated. The crude product was purified by column chromatography (PE:EA=5:1˜1:1) to give about 80 mg of the target product in colorless oil with a yield of 29%. LCMS m/z: [M+H]⁺=501.1 Synthesis of 16 (Step C)

200 mg of 15 was weighed and added into a 100 ml single-neck round bottom flask. Then 10 ml of EtOH and 5 ml of EA were added with complete dissolution. Then 40 mg of palladium carbon was added to the reaction system under nitrogen atmosphere, and the reaction system was purged with hydrogen gas for three times. The reaction system was kept under hydrogen atmosphere and stirred for 0.5 hr at room temperature. Samples were taken and detected by TLC (DCM/MeOH=10:1) to monitor the reaction. The raw material basically disappeared, and a new point was detected.

The reaction system was filtered, and the filter cake was washed with EA for 3 times. The filtrates were combined and concentrated to dryness to give 200 mg product in white solid with 100% yield. The product can be directly used in the next reaction without purification.

LCMS m/z: [M−H]⁻=409.4.

### Synthesis of 21 (Step D)

### Step D-1

2.0 g of dichlororesin was weighed and placed in a polypeptide synthesis tube. DCM (10 ml) was added and swelled at room temperature for 30 minutes. The solvent was removed by vacuum suction. The resin was washed twice with DCM, with a volume of 7 mL and a time length of 1 minute for each wash. The solvent was removed by vacuum suction. Then 16 (200 mg) was weighed and added into a 50 ml centrifuge tube. DCM (about 10 ml) was added. the solid was dissolved by shaking. Added to the above resin. Stirring was conducted to soak all the resin in the solution (if there was resin attached to the tube wall, a small amount of DCM was used to wash the tube wall). Stirring was conducted for 4-5 hours. After the reaction was complete, an appropriate amount of methanol was added. Stirring was conducted for 30 min. The solvent was removed by vacuum suction. The resin was washed with DMF once, methanol once, DMF once, methanol once and DMF twice in sequence, with a volume of 10 mL and a time length of 1 minute for each wash. The solvent was removed by vacuum suction. A small amount of dry resin was taken for ninhydrin detection. The resin was colorless and transparent, and the solution was yellowish, indicating qualified for the next coupling step.

### Step D-2

The deprotection was conducted twice by adding 10 mL readymade 20% piperidine/DMF solution and reacting for 10 minutes for each time. After the reaction was complete, the solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. Both the resin and solution were dark blue.

To a 50 mL centrifuge tube was added 563 mg Fmoc-Phe-OH, 197 mg HOBt. Then about 7 mL DMF was added. The solid was dissolved by shaking. Then 0.24 mL DIC was added. Activated for 10-30 minutes to give the activated reaction solution.

3 molar equivalent of activated reaction solution added to the resin. Stirring was conducted to soak the resin completely in the solution (if there was resin attached to the tube wall, a small amount of DCM was used to wash the tube wall). Stirring was conducted for 2-3 hours. After the reaction was complete, the solvent was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection.

The resin was colorless and transparent, and the solution was yellowish, indicating qualified for the next coupling step.

### Step D-3

The deprotection was conducted twice by adding 10 mL readymade 20% piperidine/DMF solution and reacting for 10 minutes for each time. After the reaction was complete, the solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. Both the resin and solution were dark blue.

To a 50 mL centrifuge tube was added 531 mg Fmoc-GG-OH, 197 mg HOBt. Then about 10 mL DMF was added. The solid was dissolved by shaking. Then 0.24 mL DIC was added. Activated for 10-30 minutes to give the activated reaction solution.

3 molar equivalent of activated reaction solution was added to the resin. Stirring was conducted to soak the resin completely in the solution (if there was resin attached to the tube wall, a small amount of DCM was used to wash the tube wall). Stirring was conducted for 2-3 hours. After the reaction was complete, the reaction solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. The resin was colorless and transparent, and the solution was yellowish, indicating qualified for the next coupling step.

### Step D-4

The deprotection was conducted twice by adding 10 mL readymade 20% piperidine/DMF solution and reacting for 10 minutes for each time. After the reaction was complete, the solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. Both the resin and solution were dark blue. Then, 462 mg MC-OSu was placed in a 50 mL centrifuge tube, about 10 mL DMF was added. The solid was dissolved by shaking. Then 0.24 mL DIEA was added to the resin. Stirring was conducted to soak the resin completely in the solution (if there was resin attached to the tube wall, a small amount of DCM was used to wash the tube wall). Stirring was conducted for 2-3 hours. After the reaction was complete, the reaction solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. The resin was colorless and transparent, and the solution was yellowish, indicating qualified for the next coupling step.

### Step D-5

The resin was washed twice with 10 mL of methanol. Then the solvent was removed thoroughly by vacuum suction. The resin was poured out and weighed. The lysis buffer was prepared in a 250 mL conical flask, wherein: the ratio of TFE/DCM was 80%/20%, and the volume was 7-8 times of the weight of peptide resin. The lysis buffer was added into the peptide resin, shaken well. The resin was fully soaked in the lysis buffer, and lysis was carried out at room temperature for 2-3 hours. The lysis buffer was then filtered out using a simple filter made of a syringe, and the resin was washed with 1-2 ml DCM and discarded. Then 150 mL precooled anhydrous ether was added to the lysis buffer, shaken well and then stood for 20-30 minutes. Using a 50 mL centrifuge tube, the above system was centrifuged in a centrifuge at 3500 rpm for 3 minutes, and the supernatant was poured out and discarded. The solid was shaken with precooled anhydrous ether, washed once under ultrasound, centrifuged at 3500 rpm for 3 minutes, and the supernatant was poured out and discarded. The solid was placed in a centrifuge tube and allowed to air dry overnight, and then subjected to preparative purification to give 125 mg of product in white solid with a yield of 40%. LCMS m/z: [M−H]⁻=641.5.

### Synthesis of 22 (Step E)

150 mg of raw material 21 and 55 mg of TSTU were weighed and added into a 10 mL single-neck round bottom flask, and anhydrous DMF (3 mL) was added under nitrogen atmosphere and stirred for 20 min. Then 18 mg 12-1 and 20 μl DIEA were added in sequence to the reaction system. Stirring was conducted at room temperature for 2-8 hr under nitrogen atmosphere. Samples were taken and detected by HPLC to monitor the reaction. The raw material peak completely disappeared, and new peaks were detected.

The reaction system was subjected to preparative purification, and the target product was collected and lyophilized to give about 22 mg of product in yellowish solid. LCMS m/z: [M+H]⁺=1081.0.

### Synthesis of Linker-Payload 2 (Step F)

22 (30 mg) was weighed and added into a 10 ml single-neck round bottom flask, purified water (2 ml) was added. Stirring was conducted for dissolving. DMF solution (2 ml) containing Linker-payload intermediate 1 (19.5 mg) was added to the reaction system and stirred. After reacting overnight, HPLC was used to monitor the reaction until all of the raw material had converted into intermediates. The reaction mixture was directly added with an appropriate amount of Tris Base solution or other solution that promotes the ring-opening reaction, and the reaction was performed at 0-40° C. for another 0.2-20h. The reaction was monitored by HPLC until all the intermediates were consumed and then quenched by acetic acid solution.

The reaction system was subjected to preparative purification, and the target product was collected and lyophilized to give about 25 mg of linker-payload 2 with yellowish solid.

LCMS m/z: [(M+3H)/3]⁺=1194.4

### Example 3 Construction of Antibody and the ADC

### 1. Production of the Anti-FGFR3 Antibody

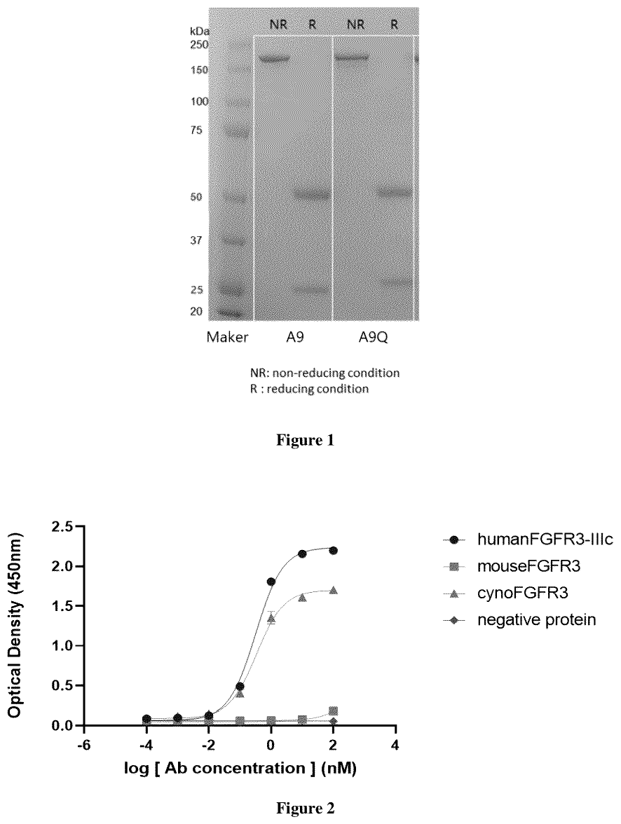

The anti-FGFR3 antibody consists of two vectors as heavy and light chains, respectively, in each mammalian expression system. Anti-FGFR3 antibody was produced using the Expi 293 transient mammalian expression system (Gibco, A14635, Carlsbad, CA, USA) via co-transfection of the above-mentioned vector. After transfection, culture supermatants were purified using the ÄKTA protein purification system (GE Healthcare Life Sciences, Uppsala, Sweden) with HiTrap Mabselect SuRe (GE Healthcare Life Sciences, 11-0034-93, Uppsala, Sweden). After purification, concentration was performed with an Amicon® Ultra Centrifugal Filter (Merck Millipore, MA, USA). The characteristics of the high-purity antibody were analyzed using SDS-PAGE (see FIG. 1) and SEC-HPLC, which show that the purity of the obtained antibody is more than 98.5%.

The anti-FGFR3 antibodies thus obtained are shown in the table below. The CDRs are highlighted with underlines and the constant regions are shown in italic.

TABLE 2

| | Heavy or | |
|---|---|---|
| Name | light chain | Amino acids Sequence |
| A9 | Heavy chain | EVQLLESGGGLVQPGGSLRLSCAASGFTFSSYDMSWVRQAPGKGLEWVSGIYSGD |
| | (SEQ ID | GSIYYADSVKGRFTISRDNSKNTLYLQMNSLRAEDTAVYYCAKDGPMNEESRFDY |
| | NO: 12) | WGQGTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQPRE |
| | | PQVYTLPPSREEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| | Light chain | QSVLTQPPSASGTPGQRVTISCSGSSSNIGNNYVSWYQQLPGTAPKLLIYADSKRPSG |
| | (SEQ ID | VPDRFSGSKSGTSASLAISGLRSEDEADYYCASWDYSLSGYVFGGGTKLTVLGQPK |
| | NO: 14) | AAPSVTLFPPSSEELQANKATLVCLISDFYPGAVTVAWKADSSPVKAGVETTTPSKQSNNK |
| | | YAASSYLSLTPEQWKSHRSYSCQVTHEGSTVEKTVAPTEC |
| A9Q | Heavy chain | EVQLLESGGGLVQPGGSLRLSCAASGFTFSSYDMSWVRQAPGKGLEWVSGIYSGD |
| | (SEQ ID | GSIYYADSVKGRFTISRDNSKNTLYLQMNSLRAEDTAVYYCAKDGPMNEESRFDY |
| | NO: 12) | WGQGTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQPRE |
| | | PQVYTLPPSREEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| | Light chain | QSVLTQPPSASGTPGQRVTISCSGSSSNIGNNYVSWYQQLPGTAPKLLIYADSKRPSG |
| | (SEQ ID | VPDRFSGSKSGTSASLAISGLRSEDEADYYCASWDYSLSGYVFGGGTKLTVLGQPK |
| | NO: 16) | AAPSVTLFPPSSEELQANKATLVCLISDFYPGAVTVAWKADSSPVKAGVETTTPSKQSNNK |
| | | YAASSYLSLTPEQWKSHRSYSCQVTHEGSTVEKTVAPTECGALPETGG |

Note:
the upstream peptide bond of GG in the LPETGG sequence is cleaved by Sortase A, and the resulting intermediate is linked to the free N-terminal of G3 to generate a new peptide bond.

### 2. SPR Analysis

The binding affinity (KD value) of the anti-FGFR3 antibody was measured using a Biacore 3000. Human (FGFR3-IIIb and -IIIc, R&D systems, 1264-FR-050), mouse (R&D systems, 710-MF-050) and cynomolgus FGFR3 proteins (Sino Biological, 90313-C02H) were coated with an amine coupling kit (GE Healthcare Life Sciences, BR100050, Uppsala, Sweden). The KD (Ka and Kd) values were evaluated according to the concentration, as shown in the following table.

TABLE 3

A9

| Antigen | Species | Ka | Kd | KD |
|---|---|---|---|---|
| FGFR3-IIIb | Human | 1.76E+6 | 1.04E−3 | 5.9E−10 |
| FGFR3-IIIc | Human | 1.67E+6 | 5.18E−4 | 3.09E−10 |
| FGFR3 | Monkey | 1.56E+6 | 1.16E−3 | 7.55E−10 |
| FGFR3 | Mouse | N.B | N.B | N.B |

N.B means antibody does not bind to FGFR3.

### 3. ADC Preparation

The linker-payload intermediates were respectively conjugated to an antibody in a site-specific manner by a ligase to form an ADC. The method for conjugation reaction can be found in WO2015165413A1. The resulting ADCs are as listed in the following table.

TABLE 4

| Name of ADC | Linker-Payload | Antibody |
|---|---|---|
| ADC19 | Linker-Payload 1 | A9Q |
| ADC20 | Linker-Payload 2 | A9Q |

### 4. Characterization of the ADC

### 1) SEC-HPLC

Chromatographic column: TSKgel G3000SWXL 7.8 mm I.D.*30 cm, 5 μm; Mobile phase: 2*PBS:methanol=9:1 (V/V); Gradient: 100% isocratic, flow rate: 1.0 ml/min; the running time was 15 min, and 280 nm was selected as the detection wavelength to analyze and detect the purity of ADC drugs.

### 2) HIC-HPLC

Chromatographic column: Proteomix HIC Butyl-NP5 4.6*100 mm, 5 μm Non-Porous; Mobile phase: 1.5M ammonium sulfate+20 mM phosphate buffered saline (pH 7.0) as mobile phase A, 20 mM phosphate buffered saline (pH 7.0): isopropanol=7:3 (V/V) as mobile phase B; Flow rate: 0.8 ml/min; phase B increased from 15% to 100% within 8 minutes; 280 nm was selected as the detection wavelength to detect the DAR distribution and calculate the averaged DAR values of ADC drugs.

The results are shown in Table 5.

TABLE 5

| | | | Residual | |
|---|---|---|---|---|
| | Purity | Averaged DAR | | Endotoxin |
| Sample name | SEC (%) | HIC | free drug | (Eu/mg) |
| ADC19 | 97.50 | 3.62 | <50 ppm | 0.09 |
| ADC20 | 95.99 | 3.58 | <50 ppm | 0.11 |

Table 5 shows that the residual free drug for both samples are lower than 50 ppm, indicating that the fall-off of the payload (cytotoxin) is very low.

### Example 4 Affinity ELISA Analysis

1) One microgram per milliliter of each human FGFR3 (R&D systems, 1264-FR-050), cynomolgus FGFR3 (Sino Biological, 90313-C₀₂H) protein or mouse FGFR3 (R&D systems, 710-MF-050) was coated on 96-well plates at 4° C. for over-night, respectively. The plates were blocked in 3% skim milk containing anti-FGFR3-antibodies and incubated for 1 h at room temperature. After washing with PBST (0.1%), the anti-human Fab antibody conjugated horseradish peroxidase (HRP) (Thermo Scientific, 31482, Waltham, MA, USA) was added at a ratio of 1:3000. Following the wash, the plate was treated with TMB solution (Thermo Scientific, N301, Waltham, MA, USA) as an HRP substrate, and the reaction was stopped with STOP solution (Cell Signaling Technology, #7002, Danvers, MA, USA).

The absorbance for each well was detected at 450 nm wavelength.

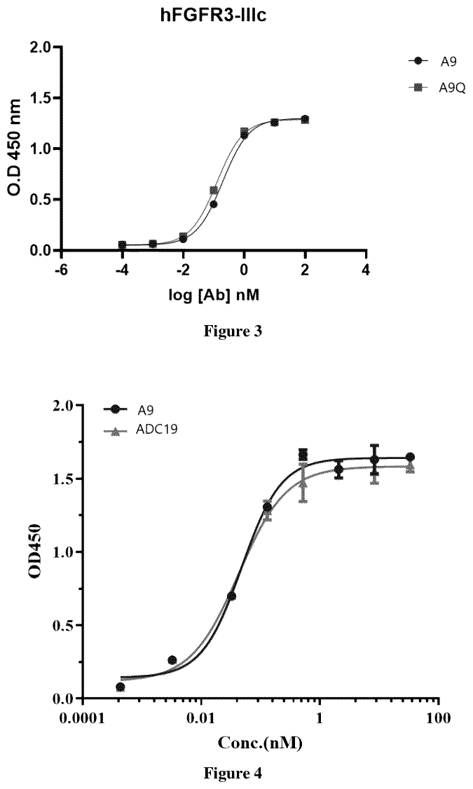

The results of this analysis are shown in FIGS. 2-3. FIG. 2 shows that the antibody A9 has high specificity to FGFR3 in both human and cynomolgus. FIG. 3 shows that A9 and A9Q have similar affinity to human FGFR3.

### 2) Affinity of ADC

The affinity of ADC binding to human FGFR3 (Sinobiological, 16044-H08H) is testing by ELISA (similar to the above method).

FIG. 4 shows that the formation of the ADC does not substantially affect the efficacy of the antibody (the EC50 of the antibody A9 and the ADC ADC19 are 0.04772 nM and 0.04272 nM, respectively).

### Example 5 Cell Binding Analysis of the Antibodies

1) The cell surface binding efficiency of anti-FGFR3 antibodies were analyzed using flow cytometry. About 3.0×10⁵ to 5.0×10⁵ FGFR3-overexpressing or FGFR3 non-expression cells were incubated with anti-FGFR3 antibodies at 4° C. for 1 h. After washing twice with Flow Cytometry Staining Buffer, the cells were stained with the goat anti-human IgG cross-adsorbed secondary antibody conjugated with Alexa Fluor 488 (Invitrogen, A-11013, Carlsbad, CA, USA) diluted 1:200 in staining buffer at 4° C. for 30 min. Mean fluorescence intensity was analyzed by flow cytometry.

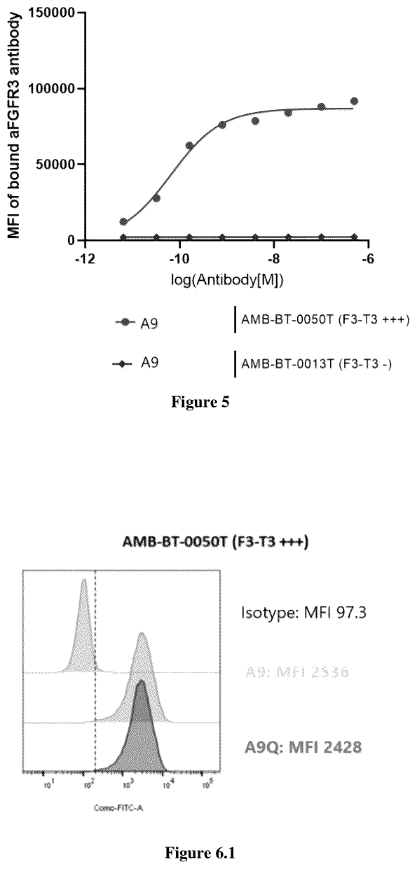

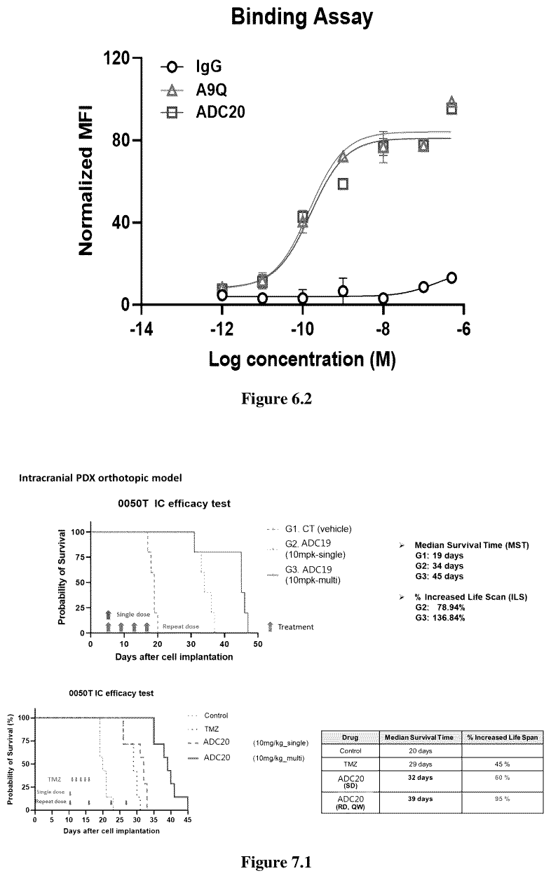

The results of this analysis are shown in FIGS. 5 and 6.1, wherein AMB-BT-0050T is a cell with positive FGFR3 expression, while AMB-BT-0013T is a cell with negative FGFR3 expression. AMB-BT-0050T, AMB-BT-0013T is sampled from patients suffering brain cancer (glioblastoma).

FIG. 5 shows that the antibody A9 has significantly higher binding affinity to the FGFR3 positive cells than to FGFR3 negative cells, indicating the antibody is highly specific for FGFR3. FIG. 6.1 shows that A9 and A9Q both bind to AMB-BT-0050T cells.

2) The cell surface binding efficiency of ADC20 was analyzed using flow cytometry. 1.0×10⁵ FGFR3-overexpressed multiple myeloma KMS-11 cells (JCRB, JCRB1178) were incubated with serial concentration of ADC20 at 4° C. for 1 h. After washing twice with Flow Cytometry Staining Buffer, the cells were stained with the goat anti-human IgG cross-adsorbed secondary antibody conjugated with Alexa Fluor 647 (Invitrogen, A-21445, Carlsbad, CA, USA) diluted 1:300 in staining buffer at 4° C. for 30 min. Antibody A9Q and Human IgG1 kappa Isotype (CrownVivo, C0001) as control. Mean fluorescence intensity was analyzed by flow cytometry.

The results of this analysis are shown in FIG. 6.2, shows that ADC20 and antibody A9Q have similar and significantly high binding affinity to KMS-11.

### Example 6 In Vitro Cytotoxicity Assay

1) 3D single spheroid model was formed by isolating cancer cells derived from glioblastoma patients. Two types of patient-derived cells with FGFR3 overexpression (AMB-BT-0050T, AMB-BT-0112T) and FGFR3 non-expression derived cells (AMB-BT-0013T), sampled from patients suffering from glioblastoma, were incubated overnight to form single spheroids (3D), followed by ADC was treated and incubated for a week, and then spheroid size and volume were quantified. The results are shown in the table below, indicating that for both the conjugates ADC19 and ADC20, the cytotoxicity on FGFR3 positive cells are significantly higher than that to FGFR3 negative cells, and thus the ADCs are highly specific for FGFR3.

TABLE 6.1

| | |
|---|---|
| FGFR3 | IC50 (nM) |

| Cell | Type | expression | ADC19 | ADC20 |
|---|---|---|---|---|
| AMB-BT- | GBM | +++ | 1 | 0.4 |
| 0050T | PDCs | | | |
| AMB-BT- | | ++ | 10 | 21 |
| 0112T | | | | |
| AMB-BT- | | − | 400 | 700 |
| 0013T | | | | |

GBM is abbreviation of glioblastoma, PDCs is abbreviation of patient-derived cells.

2) 3D single spheroid model was formed from bladder cell line RT112 (DSMZ, ACC418), which is FGFR3 overexpression. RT112 were seeded in cell spheroid culture plate and incubated overnight to form single spheroids (3D), followed by ADC was treated and incubated for a week, and then spheroid size and volume were quantified. The results are shown in the table 6.2 below, indicating that for both the conjugates ADC19 and ADC20 had significantly cytotoxicity on FGFR3 positive bladder cancer cells.

TABLE 6.2

| | |
|---|---|
| FGFR3 | IC50 (nM) |

| Cell | Type | expression | ADC19 | ADC20 |
|---|---|---|---|---|
| RT112 | Bladder | ++ | 227 | 10 |

### Example 7 In Vivo Efficacy Test in Glioblastoma PDX Models

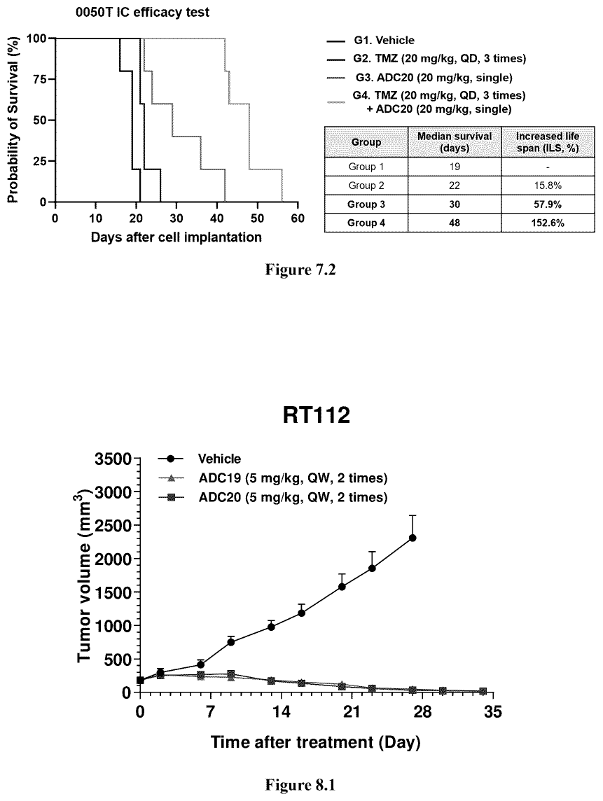

1) For evaluating the survival rate in an orthotopic mouse model of brain tumor with target expression, AMB-BT-0050T patient-derived cells were sub-cultured and mixed 2.0×10⁵ cells with the medium. 7-wk-old female BALB/c nude mice were used for intracranial transplantation. The prepared patient-derived cells were injected into the brains of mice by stereotactic intracranial injection at a depth of 3.2 mm at a position of 1.7 mm left and 0.5 mm above the bregma. Mice were housed with a 12-h light/12-h dark cycle and ad libitum access to food and water. Therapeutics administration is as follows. TMZ (temozolomide) was injected through oral administration every day for 5 times. ADCs were administered only once (single injection) or once a week for four weeks (multi-injection) via intravenous injection from the 7^{th} day after model production to each group. The mice were sacrificed either when 20% body weight loss or neurological symptoms (lethargy, ataxia, and seizures) were observed and the results are shown in FIG. 7.1. The survival of mice is evaluated through MST (Median Survival Time) and ILS (Increase Life Span); MST, the time point at which the probability of survival equals 50%; ILS (Increase Life Span), ILS (%)=[(median survival time of treated group)/(median survival time of control group)−1]×100.

The results show that anti-FGFR3 ADCs could inhibit the progression of brain tumors and prolong the survival time.

2) To furtherly evaluate the survival rate in an orthotopic mouse model of brain tumor with target expression, in AMB-BT-0050T PDX model, mice were grouped and treated with (1) vehicle, (2) TMZ (temozolomide), 20 mg/kg; (3) ADC20 20 mg/kg; (4) combination of TMZ and ADC20. TMZ was injected through oral administration every day for 3 times. ADCs were administered once via intravenous injection. The results are shown in FIG. 7.2. The survival of mice is evaluated through MST and ILS.

The results show both monotherapy of ADC20 and combo of ADC20 with TMZ could inhibit the progression of brain tumors and prolong the survival time. Combination of ADC and TMZ (standard of care for GBM) can significantly prolonger the survival time.

### Example 8 In Vivo Efficacy Test in Bladder Cancer CDX Models

To evaluate the in vivo anti-tumor efficacy of ADC19 and ADC20 in mice bearing bladder cancer FGFR3-high CDX model, several types of FGFR3 overexpressed bladder cancer CDX models were used.

8.1) RT112 cells in exponential growth stage were collected and counted for tumor inoculation. 0.2 mL of matrix gel buffer (PBS:Matrigel=1:1) was used to subcutaneously inject 10×10⁶ cells into the right flank of SPF female BALB/c nude mice aged 6-8 weeks.

The tumor diameter was measured with a caliper and the tumor volume was calculated according to the formula V=0.5 a×b² (wherein a is the long diameter of the tumor and b is the short diameter of the tumor). When the mean tumor volume was about 100-300 mm³, the mice were randomized into vehicle group, ADC19 5 mg/kg group and ADC20 5 mg/kg group. The day of first administration is defined as day 0. Mice in the vehicle group were given the solvent of ADC drugs with the same frequency and administration route. The tumor volume of mice in each group was measured twice a week. The experiment was end on day 27, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

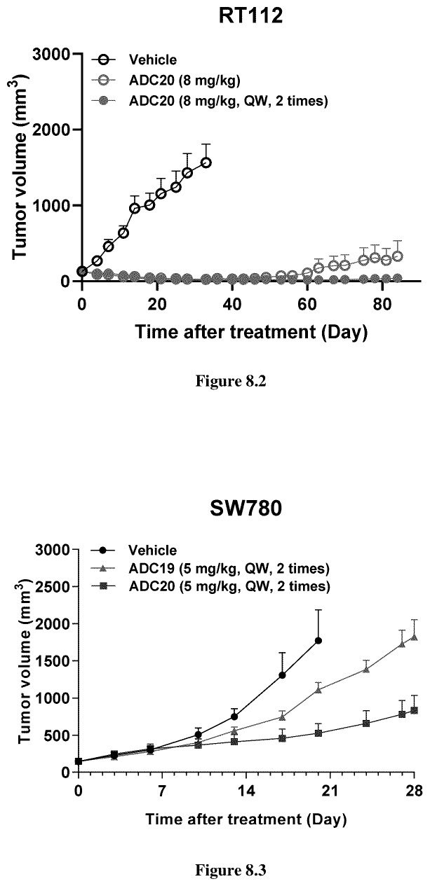

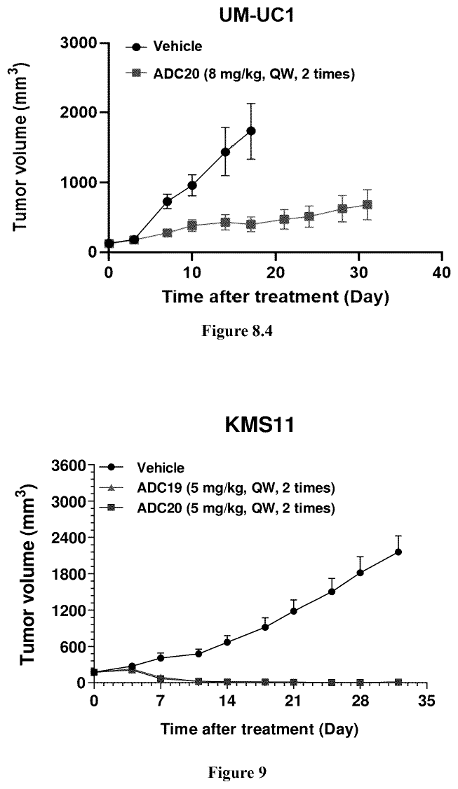

FIG. 8.1 showed the tumor volume change of tumor bearing BALB/c nude mice treated with: (1) vehicle; (2) ADC19 5 mg/kg, QW, 2 times; (3) ADC20 5 mg/kg, QW, 2 times. Table 7.1 showed on the end day (day 27), the mean tumor volumes of ADC19 5 mg/kg group, ADC20 5 mg/kg group were 48 mm³ and 32 mm³ respectively; TGI were 106.35% and 107.08% respectively.

TABLE 7.1

| | | TGI^{b} |
|---|---|---|
| Group | Tumor volume (mm³) ^{a} | (%) |
| Vehicle | 2,307 ± 336 | — |
| ADC19 5 mg/kg | 48 ± 9 | 106.35 |
| ADC20 5 mg/kg | 32 ± 7 | 107.08 |

^{a} Mean ± SEM; measured on the end day;
^{b}TGI (%) = [1 − (T₂₇ − T₀)/(V₂₇ − V₀)] × 100%. T₀ is the mean tumor volume of the treatment group on the first day of administration, and T₂₇ is the mean tumor volume of the treatment group at day 27 after administration; V₀ is the mean tumor volume of the vehicle group on the first day of administration, V₂₇ is the mean tumor volume of the vehicle group at the day 27 after administration.

The results show both ADC19 and ADC20 have excellent anti-tumor efficacy in FGFR3 overexpressed RT112 bladder cancer CDX model.

8.2) For evaluation the dose-dependent antitumor efficacy in bladder cancers, the RT112 cells bearing BALB/c nude mice were randomized grouped and treated by solvent; ADC20 8 mg/kg single dose and ADC20 8 mg/kg, QW, 2 times. The experiment was end on day 33, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%. The ADC groups continued to be observed until day 84.

FIG. 8.2 showed the tumor volume change of tumor bearing BALB/c nude mice treated with: (1) vehicle; (2) ADC20 8 mg/kg, single dose; (3) ADC20 8 mg/kg, QW, 2 times. Table 7.2 showed on the end day (day 33), the mean tumor volumes of ADC20 single dose group, ADC20 repeat dose group were 36 mm³ and 16 mm³ respectively; TGI were 106.34% and 107.65% respectively. And cause completed response (CR) in repeated dose group.

TABLE 7.2

| | | TGI |
|---|---|---|
| Group | Tumor volume (mm³) | (%) |
| Vehicle | 1,564 ± 244 | — |
| ADC20 8 mg/kg, single | 36 ± 45 | 106.34 |
| ADC20 8 mg/kg, repeat | 16 ± 16 | 107.65 |

The results show either single dose or repeated dose of ADC20 have excellent anti-tumor efficacy in FGFR3 overexpressed RT112 bladder cancer CDX model, and repeated dose of ADC20 caused CR in this bladder cancer CDX model.

8.3) SW780 cells in exponential growth stage were collected and counted for tumor inoculation. 0.2 mL of matrix gel buffer (PBS:Matrigel=1:1) was used to subcutaneously inject 10×10⁶ cells into the right flank of SPF female NOD SCID mice aged 6-8 weeks.

The tumor diameter was measured with a caliper and the tumor volume was calculated according to the formula V=0.5 a×b². When the mean tumor volume was about 100-300 mm³, the mice were randomized into vehicle group, ADC19 5 mg/kg group and ADC20 5 mg/kg group. The day of first administration is defined as day 0. Mice in the vehicle group were given the solvent of ADC drugs with the same frequency and administration route. The tumor volume of mice in each group was measured twice a week. The experiment was end on day 20, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

FIG. 8.3 showed the tumor volume change of tumor bearing NOD SCID mice treated with: (1) vehicle; (2) ADC19 5 mg/kg, QW, 2 times; (3) ADC20 5 mg/kg, QW, 2 times. Table 7.3 showed on the end day (day 20), the mean tumor volumes of ADC19 5 mg/kg group, ADC20 5 mg/kg group were 1,109 mm³ and 526 mm³ respectively; TGI were 40.80% and 76.66% respectively.

TABLE 7.3

| | | TGI |
|---|---|---|
| Group | Tumor volume (mm³) | (%) |
| Vehicle | 1,771 ± 414 | — |
| ADC19 5 mg/kg | 1,109 ± 100 | 40.80 |
| ADC20 5 mg/kg | 526 ± 128 | 76.66 |

The results show both ADC19 and ADC20 have potential anti-tumor efficacy in FGFR3 overexpressed SW780 bladder cancer CDX model.

8.4) UM-UC1 cells in exponential growth stage were collected and counted for tumor inoculation. 0.1 mL of matrix gel buffer (PBS:Matrigel=1:1) was used to subcutaneously inject 10×10⁵ cells into the right flank of SPF female Balb/c nude mice aged 6-8 weeks.

The tumor diameter was measured with a caliper and the tumor volume was calculated according to the formula V=0.5 a×b². When the mean tumor volume was about 100-300 mm³, the mice were randomized into vehicle group and ADC20 8 mg/kg group. The day of first administration is defined as day 0. Mice in the vehicle group were given the solvent of ADC drugs with the same frequency and administration route. The tumor volume of mice in each group was measured twice a week. The experiment was end on day 17, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

FIG. 8.4 showed the tumor volume change of tumor bearing Balb/c nude mice treated with: (1) vehicle; (2) ADC20 8 mg/kg, QW, 2 times. Table 7.4 showed on the end day (day 17), the mean tumor volume of ADC20 8 mg/kg group was 402 mm³; TGI was 82.71%.

TABLE 7.4

| | | TGI |
|---|---|---|
| Group | Tumor volume (mm³) | (%) |
| Vehicle | 1,735 ± 394 | — |
| ADC20 8 mg/kg | 402 ± 104 | 82.71 |

The results show that ADC20 has anti-tumor efficacy in FGFR3 overexpressed UM-UC1 bladder cancer CDX model.

### Example 9 In Vivo Efficacy Test in Multiple Myeloma CDX Models

To evaluate the in vivo anti-tumor efficacy of ADC19 and ADC20 in mice bearing multiple myeloma FGFR3 overexpressed CDX model, KMS11 cells in exponential growth stage were collected and counted for tumor inoculation. 0.2 mL of matrix gel buffer (PBS:Matrigel=1:1) was used to subcutaneously inject 10×10⁶ cells into the right flank of SPF female CB17.SCID mice aged 6-8 weeks.

The tumor diameter was measured with a caliper and the tumor volume was calculated according to the formula V=0.5 a×b². When the mean tumor volume was about 100-300 mm³, the mice were randomized into vehicle group, ADC19 5 mg/kg group and ADC20 5 mg/kg group. The day of first administration is defined as day 0. Mice in the vehicle group were given the solvent of ADC drugs with the same frequency and administration route. The tumor volume of mice in each group was measured twice a week. The experiment was end on day 32, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

FIG. 9 showed the tumor volume change of tumor bearing CB17.SCID mice treated with: (1) vehicle; (2) ADC19 5 mg/kg, QW, 2 times; (3) ADC20 5 mg/kg, QW, 2 times. Table 8 showed on the end day (day 32), the mean tumor volumes of ADC19 5 mg/kg group, ADC20 5 mg/kg group were 9 mm³ and 11 mm³ respectively; TGI were 108.41% and 108.31% respectively.

TABLE 8

| | | TGI |
|---|---|---|
| Group | Tumor volume (mm³) | (%) |
| Vehicle | 2,161 ± 263 | — |
| ADC19 5 mg/kg | 9 ± 0 | 108.41 |
| ADC20 5 mg/kg | 11 ± 1 | 108.31 |

The results show both ADC19 and ADC20 have excellent anti-tumor efficacy in FGFR3 overexpressed KMS11 multiple myeloma CDX model.

### Example 10 Internalization Activity of ADC

Multiple myeloma cells KMS-11 (JCRB, JCRB1178) in good viability, were treated with accutase, collected, suspended in serum-free RPMI medium. 1.0×10⁵ cells were dyed with 1000 times diluted LIVE/DEAD™ Fixable Near-IR Dead Cell Stain dye for 30 min at RT in dark. And washed twice by cold FACS buffer to remove the excess dye and cells were incubated with tested drugs solution with the final concentration of 1 nM for 30 min on ice. After the surface binding, the antibody-cell mixture was washed by cold FACS buffer and serum-free RPMI medium to remove the excess antibody. Then antibody-bound cells were incubated at 37° C. CO₂ incubator for 0 min, 10 min, 30 min, 1 h, 2 h and 4h, respectively, for antibody internalization. After incubation the antibody bonded cells were washed with Flow Cytometry Staining Buffer and fluorescence labeled by ice cold goat anti-human-IgG-IgG cross-adsorbed secondary antibody conjugated with Alexa Fluor 488 (Invitrogen, A-11013, Carlsbad, CA, USA) diluted 1:300 in staining buffer at 4° C. for 30 min. After the fluorescence labelling, the antibody-cell mixture was washed twice again and analyzed by flow cytometry.

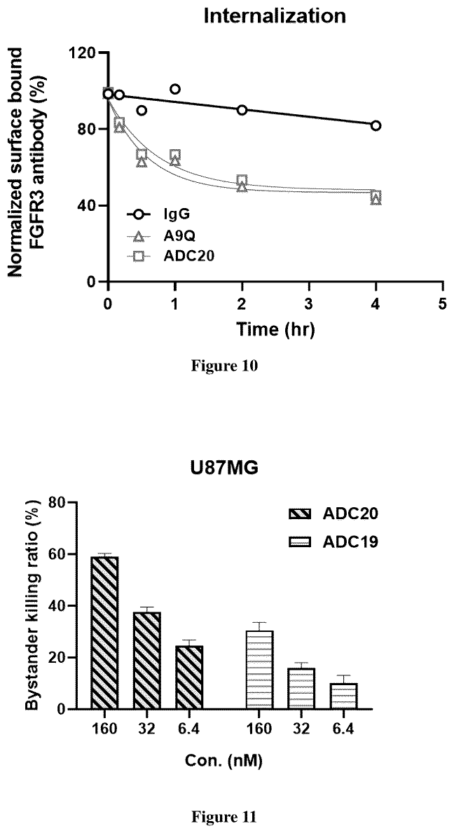

The MFI data was normalized to MFI at 0 min, which was defined as 100%. The result was analyzed by GraphPad Prism 9. As shown in FIG. 10, ADC20 shows comparable internalization activity with A9Q.

### Example 11 Bystander Killing Effect of ADC20 in RT112/U87MG

Far-Red labelled FGFR3-positive RT112 cells and CFSE labelled FGFR3-negative U87MG cells were seeded onto 96 well round bottom plate with 1.0×10⁴ cells for each cell types. The cells were incubated overnight for stable adhesion to plate, followed by treatment with ADCs or PBS and incubation for a week. After incubation, the cells were centrifuged at 2300 rpm for 2 min, and then supernatant was discarded. The cells were washed once with PBS, detached, and resuspended into flow cytometry staining buffer containing LIVE/DEAD staining dye. Finally, the total amount of FGFR3-positive and FGFR3-negative cells and their viability were detected and analyzed by Flow cytometer (Novocyte, Agilent).

As shown in FIG. 11, ADC19 and ADC20 both have bystander killing effect, and ADC20 has a more effective bystander killing effect than ADC 19.

### Example 12 In Vivo Efficacy Test in Glioblastoma PDX Models

To evaluate the in vivo anti-tumor efficacy of ADC20 in mice bearing glioblastoma FGFR3-high PDX model, several types of FGFR3 overexpressed glioblastoma PDX models were used.

1) AMB-BT-0039T cells with FGFR3-TACC3 fusion (GBM, patients-derived cells) in exponential growth stage were collected and counted for tumor inoculation. 0.1 mL of matrix gel buffer (PBS:Matrigel=1:1) was used to subcutaneously inject 1×10⁶ cells into the right flank of SPF female BALB/c nude mice aged 6-8 weeks. The tumor diameter was measured with a caliper and the tumor volume was calculated according to the formula V=0.5 a×b² (wherein a is the long diameter of the tumor and b is the short diameter of the tumor). When the mean tumor volume was about 100-300 mm³, the mice were randomized into vehicle group, and ADC20 8 mg/kg group. The day of first administration is defined as day 0. Mice in the vehicle group were given the solvent of ADC drugs with the same frequency and administration route. The tumor volume of mice in each group was measured twice a week. The experiment was end on day 24, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

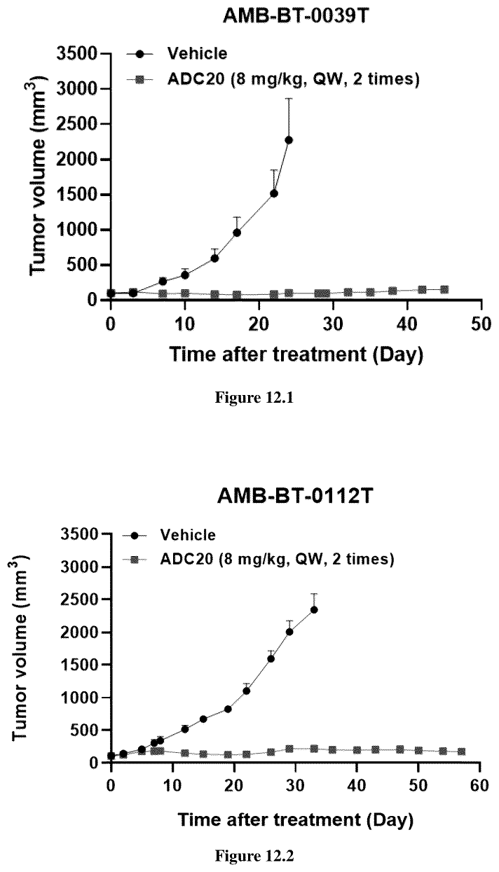

FIG. 12.1 showed the tumor volume change of tumor bearing BALB/c nude mice treated with: (1) vehicle; (2) ADC20 8 mg/kg, QW, 2 times. Table 9 showed on the end day (day 24), the mean tumor volumes of ADC20 8 mg/kg group was 100 mm³ and TGI was 100.03%.

TABLE 9

| | | TGI^{b} |
|---|---|---|
| Group | Tumor volume (mm³) ^{a} | (%) |
| Vehicle | 2,274 ± 592 | — |
| ADC20 8 mg/kg | 100 ± 18 | 100.03 |

^{a} Mean ± SEM; measured on the end day;
^{b}TGI (%) = [1 − (T₂₄ − T₀)/(V₂₄ − V₀)] × 100%. T₀ is the mean tumor volume of the treatment group on the first day of administration, and T₂₄ is the mean tumor volume of the treatment group at day 24 after administration; V₀ is the mean tumor volume of the vehicle group on the first day of administration, V₂₄ is the mean tumor volume of the vehicle group at the day 24 after administration.

2) AMB-BT-0112T cells with FGFR3-TACC3 fusion (GBM, patients-derived cells) in exponential growth stage were collected and counted for tumor inoculation. 0.1 mL of matrix gel buffer (PBS:Matrigel=1:1) was used to subcutaneously inject 1×10⁶ cells into the right flank of SPF female BALB/c nude mice aged 6-8 weeks.

The tumor diameter was measured with a caliper and the tumor volume was calculated according to the formula V=0.5 a×b² (wherein a is the long diameter of the tumor and b is the short diameter of the tumor). When the mean tumor volume was about 100-300 mm³, the mice were randomized into vehicle group, and ADC20 8 mg/kg group. The day of first administration is defined as day 0. Mice in the vehicle group were given the solvent of ADC drugs with the same frequency and administration route. The tumor volume of mice in each group was measured twice a week.

FIG. 12.2 showed the tumor volume change of tumor bearing BALB/c nude mice treated with: (1) vehicle; (2) ADC20 8 mg/kg, QW, 2 times. Table 10 showed on the end day (day 33), the mean tumor volumes of ADC20 8 mg/kg group was 216 mm³ and TGI was 95.08%.

TABLE 10

| | | TGI^{b} |
|---|---|---|
| Group | Tumor volume (mm³) ^{a} | (%) |
| Vehicle | 2,344 ± 242 | — |
| ADC20 8 mg/kg | 216 ± 52 | 95.08 |

### ACKNOWLEDGEMENT

The application is partially supported by Korea Drug Development Fund funded by Ministry of Science and ICT, Ministry of Trade, Industry, and Energy, and Ministry of Health and Welfare (HN21C0803, Republic of Korea).

1. An antibody drug conjugate (ADC) having the structure of formula (I): 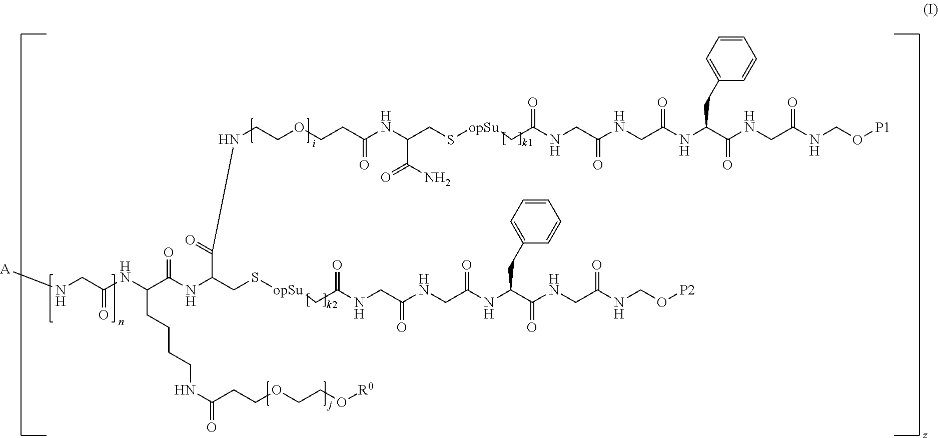
wherein,
A is an anti-FGFR3 antibody or an antigen binding fragment thereof, the antibody or antigen binding fragment is modified to connect with the (Gly)ₙ moiety in the compound of formula (I), wherein the antibody or an antigen binding fragment comprises CDRs: a heavy chain CDR1 comprising amino acid sequence of SEQ ID NO: 1 or having one to three conservative amino acid substitutions compared to SEQ ID NO: 1, a heavy chain CDR2 comprising amino acid sequence of SEQ ID NO: 2 or having one to three conservative amino acid substitutions compared to SEQ ID NO: 2, a heavy chain CDR3 comprising amino acid sequence of SEQ ID NO: 3 or having one to three conservative amino acid substitutions compared to SEQ ID NO: 3, a light chain CDR1 comprising amino acid sequence of SEQ ID NO: 4 or having one to three conservative amino acid substitutions compared to SEQ ID NO: 4, a light chain CDR2 comprising amino acid sequence of SEQ ID NO: 5 or having one to three conservative amino acid substitutions compared to SEQ ID NO: 5, and a light chain CDR3 comprising amino acid sequence of SEQ ID NO: 6 or having one to three conservative amino acid substitutions compared to SEQ ID NO: 6;
z is an integer of 1 to 20; preferably 1 to 4; particularly 2;
opSu is 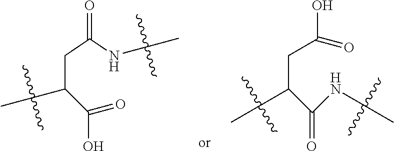 [SMILES: *NC(=O)CC(*)C(=O)O ; *NC(=O)C(*)CC(=O)O]
or a mixture thereof;
R⁰ is C₁₋₁₀ alkyl;
n is any integer of 2 to 20;
k1 and k2 are independently an integer of 1 to 7;
i is an integer of 1-100;
j is an integer of 1-100;
P1 and P2 are independently a payload.

2. The antibody drug conjugate of claim 1, wherein the connection process between the modified antibody or antigen binding fragment and the Compound of formula (I) is catalyzed by a ligase, optionally wherein the ligase is sortase.

3. The antibody drug conjugate of claim 1, wherein the payload is a cytotoxin or a fragment thereof, with an optional derivatization in order to connect the payload and linker;
the cytotoxin is selected from the group consisting of taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhy-drovinblastine, dolastatin 10 and analogues, halichondrin B, eribulin, indole-3-oxoacetamide, podophyllotoxins, 7-diethylamino-3-(2′-benzoxazolyl)-coumarin (DBC), discodermolide, laulimalide, camptothecins and derivatives thereof, mitoxantrone, mitoguazone, nitrogen mustards, nitrosoureasm, aziridines, benzodopa, carboquone, meturedepa, uredepa, dynemicin, esperamicin, neocarzinostatin, aclacinomycin, actinomycin, antramycin, bleomycins, actinomycin C, carabicin, carminomycin, cardinophyllin, carminomycin, actinomycin D, daunorubicin, detorubicin, 5zacytidin, epirubicin, esorubicin, idarubicin, marcellomycin, mitomycins, nogalamycin, olivomycin, peplomycin, porfiromycin, puromycin, ferric 5zacytidin, rodorubicin, rufocromomycin, streptozocin, zinostatin, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, tiamiprine, thioguanine, ancitabine, gemcitabine, enocitabine, 6zacytidine, 6-azauridine, carmofur, cytarabine, dideoxyuridine, doxifluridine, floxuridine, calusterone, dromostanolone propionate, epitiostanol, mepitiostane, testolactone, aminoglutethimide, mitotane, trilostane, flutamide, nilutamide, bicalutamide, leuprorelin acetate, protein kinase inhibitors and a proteasome inhibitors; and/or
selected from vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, pyrrolobenzodiazepine, exatecan and derivatives thereof, and/or
selected from auristatins, especially MMAE, MMAF or MMAD; and/or
selected from exatecan and derivatives thereof, such as DX8951f, and/or
selected from DXd-(1) and DXd-(2); preferably DXd-(1).

4. The antibody drug conjugate of claim 1, wherein the payload has the structure of formula (II): 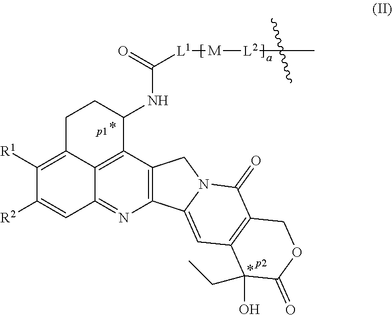
wherein,
a is 0 or 1;
the carbon atoms marked with p1* and p2* each is asymmetric center, and the asymmetric center is S configured, R configured or racemic;
L¹ is selected from C₁₋₆ alkylene, which is unsubstituted or substituted with one substituent selected from halogen, —OH and —NH₂;
M is —CH₂—, —NH— or —O—;
L² is C₁₋₃ alkylene;
R¹ and R² are each independently selected from hydrogen, C₁₋₆ alkyl, halogen and C₁₋₆ alkoxy;

5. The antibody drug conjugate of claim 1, wherein the payload is selected from:

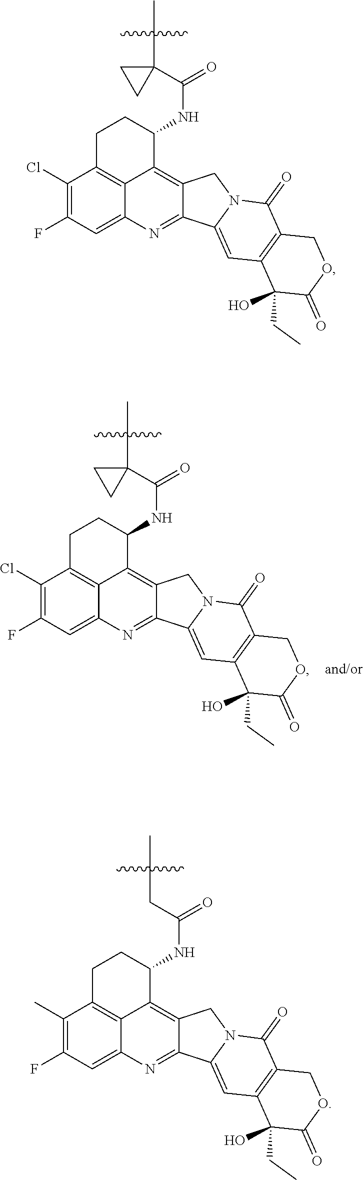 [SMILES: *C1(C(=O)N[C@H]2CCc3c(Cl)c(F)cc4nc5c(c2c34)Cn2c-5cc3c(c2=O)COC(=O)[C@]3(O)CC)CC1 ; *C1(C(=O)N[C@@H]2CCc3c(Cl)c(F)cc4nc5c(c2c34)Cn2c-5cc3c(c2=O)COC(=O)[C@]3(O)CC)CC1 ; *CC(=O)N[C@H]1CCc2c(C)c(F)cc3nc4c(c1c23)Cn1c-4cc2c(c1=O)COC(=O)[C@]2(O)CC]

6. The antibody drug conjugate of claim 1, which is selected from:

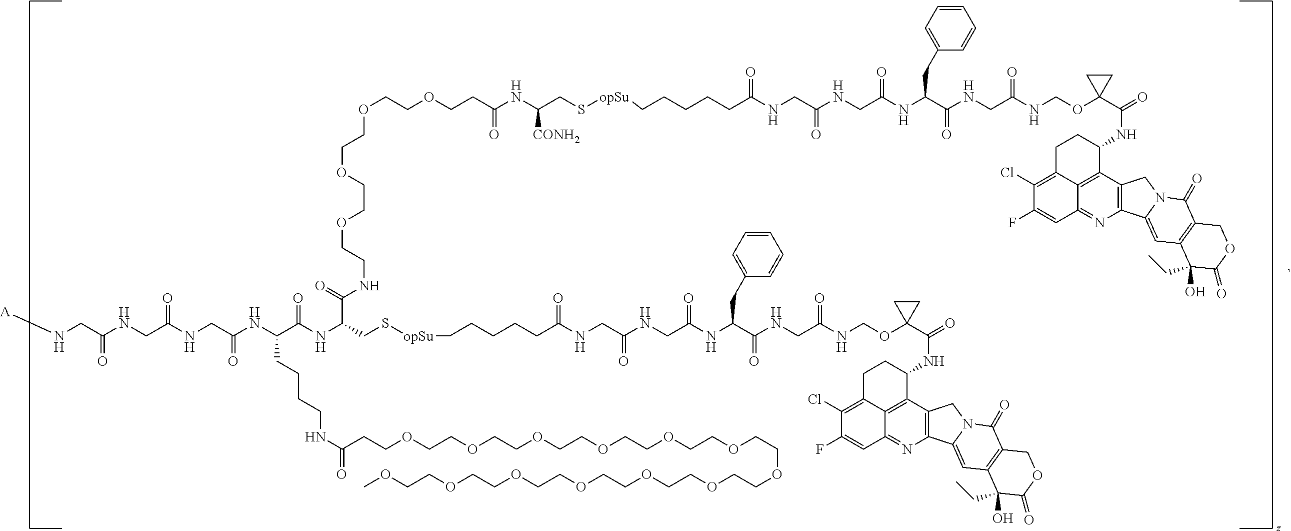

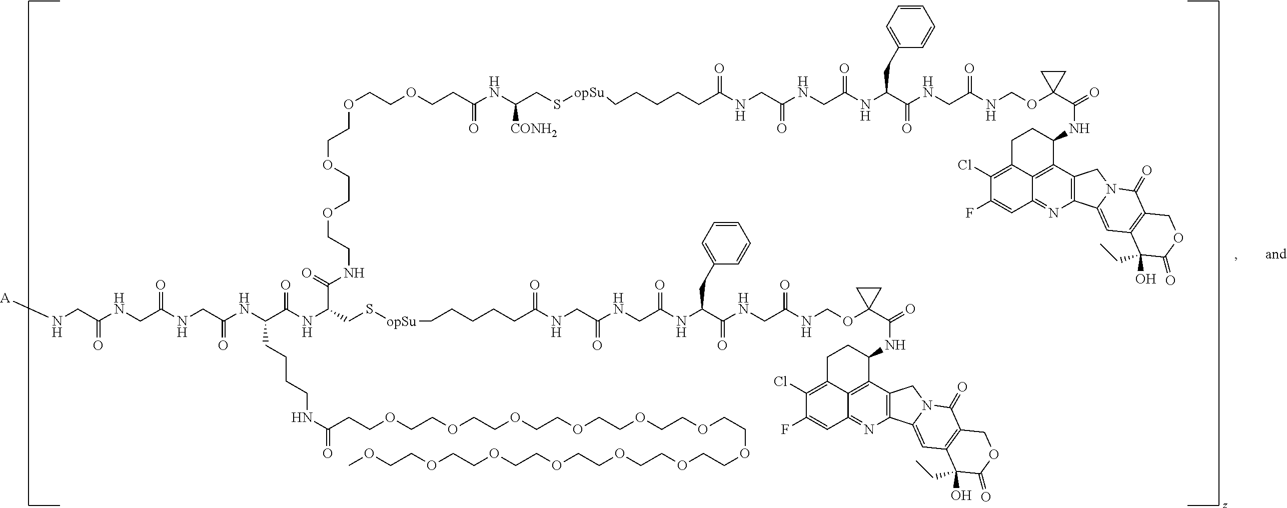

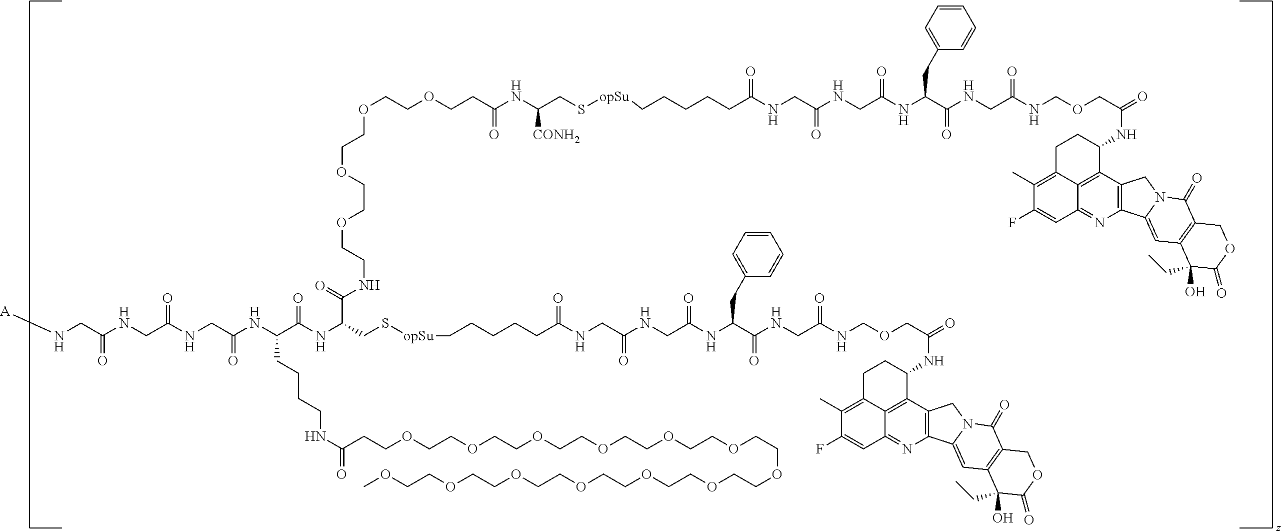

7. The conjugate of a m claim 1, wherein
the antibody or antigen binding fragment comprises a VH domain comprising an amino acid sequence having at least about 90% sequence identity to the amino acid sequence of SEQ ID NO: 7; and/or
a VL domain comprising an amino acid sequence having at least about 90% sequence identity to the amino acid sequence of SEQ ID NO:8.

8. The antibody drug conjugate of claim 1, wherein
the antibody or an antigen binding fragment comprises a heavy constant domain comprising an amino acid sequence having at least about 90% sequence identity to the amino acid sequence of SEQ ID NO: 9 or SEQ ID NO: 10; and/or
a light constant domain comprising an amino acid sequence having at least about 90% sequence identity to the amino acid sequence of SEQ ID NO:11.

9. The antibody drug conjugate of claim 1, wherein
the antibody or an antigen binding fragment comprises a heavy chain comprising an amino acid sequence having at least about 90% sequence identity to the amino acid sequence of SEQ ID NO: 12 or 13; and/or
a light chain comprising an amino acid sequence having at least 90% sequence identity to the amino acid sequence of SEQ ID NO: 14.

10. (canceled)

11. The antibody drug conjugate of claim 1, wherein the antibody or the antigen-binding fragment comprises C-terminal modification of the heavy chain and/or C-terminal modification of the light chain, the antibody, Sp and recognition sequence of the ligase donor substrate are sequentially linked; Sp is a spacer sequence selected from GA, GGGGS, GGGGSGGGGS and GGGGSGGGGSGGGGS; the recognition sequence of the ligase donor substrate is LPXTGJ, wherein X can be any single amino acid that is natural or unnatural; J is absent, or is an amino acid fragment comprising 1-10 amino acids.

12. The antibody drug conjugate of claim 1, wherein modified antibody or the antigen-binding fragment thereof comprises a heavy chain of SEQ ID NO: 15, a light chain of SEQ ID NO: 16; and/or
modified antibody or the antigen-binding fragment thereof comprises a heavy chain of SEQ ID NO: 15, a light chain of SEQ ID NO: 14; and/or
modified antibody or the antigen-binding fragment thereof comprises a heavy chain of SEQ ID NO: 12, a light chain of SEQ ID NO: 16.

13. The antibody drug conjugate of claim 1, wherein the antibody drug conjugate has a drug to antibody ratio (DAR) of an integer or non-integer of 1 to 20, particularly 2-8.

14. The antibody drug conjugate of claim 1, wherein the KD value of the anti-FGFR3 antibody or an antigen binding fragment binding to human FGFR3 and monkey FGFR3 is less than 10 nM.

15. A pharmaceutical composition comprising a prophylactically or therapeutically effective amount of the antibody drug conjugate of claim 1, and at least one pharmaceutically acceptable carrier.

16. The pharmaceutical composition of claim 15, wherein the composition comprises an alkylating agent, optionally wherein the alkylating agent comprises temozolomide or derivates thereof.

17. (canceled)

18. (canceled)

19. A kit comprising the antibody drug conjugate of claim 1.

20. The kit of claim 19, comprising
a first packaging unit, comprising the conjugate of claim 1,
a second packaging unit, comprising an alkylating agent; and
optionally an instruction for administrating the conjugate and alkylating agent to a subject.

21.-24. (canceled)

25. A method for treating a subject suffering a disease or preventing disease progression, comprises administering the antibody drug conjugate of claim 1 to the subject, and the disease is FGFR3-mediated disease, optionally wherein the disease includes tumor overexpressing FGFR3 or tumor with FGFR3 gene mutation.

26. (canceled)

27. The method of claim 25, wherein the disease is selected from: brain cancer, bladder cancer, urothelial cancer, cervical cancer, multiple myeloma or intrahepatic cholangiocarcinoma, optionally wherein the disease is glioblastoma, bladder cancer or multiple myeloma.

28. (canceled)

29. The method of claim 25, wherein the method further comprises administering an alkylating agent to the subject, wherein
the conjugate and alkylating agent are administered simultaneously as part of the same pharmaceutical formulation; or
the conjugate and alkylating agent are administered simultaneously as part of the different pharmaceutical formulation; or
the conjugate and alkylating agent are administered at different times, and optionally wherein the alkylating agent comprises temozolomide or derivates thereof.

30.-33. (canceled)
